(19) United States
(12) Patent Application Publication
(10) Pub. No.: US 2026/0130609 A1
(43) Pub. Date: May 14, 2026

(54) SELF-CALIBRATING ADAPTIVE OPTICAL SYSTEMS, DEVICES, AND METHODS FOR BIOLOGICAL ANALYTE MEASUREMENTS

(71) Applicant: Computational Imaging LLC, Louisville, CO (US)

(72) Inventors: Benjamin Braker, Louisville, CO (US); Ronald Zimmerman, Boulder, CO (US)

(21) Appl. No.: 19/386,626

(22) Filed: Feb. 3, 2026

Related U.S. Application Data
(60) Provisional application No. 63/719,502, filed on Nov. 12, 2024.
(60) Provisional application No. 63/869,087, filed on Aug. 22, 2025.

(51) Int. Cl. A61B 5/145; A61B 5/00; A61B 5/1455; A61B 5/1495

(52) U.S. Cl. CPC A61B 5/14546; A61B 5/14503; A61B 5/1455; A61B 5/1495; A61B 5/6861; A61B 5/7271; A61B 2560/0214; A61B 2560/0223; A61B 2562/0233; A61B 2562/0271; A61B 2562/12; A61B 2562/162

(57) ABSTRACT

Self-calibrating adaptive optical systems, devices, and methods for biological analyte measurements are provided in accordance with various embodiments. Some embodiments include a method that includes emitting light from a laser. The light from the laser is filtered utilizing one or more short pass filters. The filtered light from the laser is focused onto an object. Light scatters from the object. The scattered light from the object is filtered utilizing a long pass filter. The scattered light from the object is collected into a spectrometer aperture. The scattered light from the object passes through a spectrometer to spatially separate the spectral components of the scattered light into a scattered spectra; the scattered spectra is filtered and detected on one or more optical sensors to form a detected spectra. The detected spectra is digitized to deliver a Raman spectra to a processor for analysis.

### CROSS-REFERENCES TO RELATED APPLICATIONS

This application is a non-provisional patent application claiming priority benefit of U.S. provisional patent application Ser. No. 63/719,502, filed on Nov. 12, 2024 and entitled “BI-DIRECTIONAL ADAPTIVE OPTICAL SYSTEMS AND METHODS,” and U.S. provisional patent application Ser. No. 63/869,087, filed on Aug. 22, 2025 and entitled “SELF-CALIBRATION OF RAMAN SPECTRA TO ESTIMATE SITE-TO-SITE AND SUBJECT-TO-SUBJECT VARIATIONS,” the entire disclosures of which are herein incorporated by reference for all purposes.

### BACKGROUND

A wide variety of tools and techniques may be utilized with respect to blood analysis for medical, health, or other purposes. The tools and techniques, however, may involve a blood draw. There may be a need for new tools and techniques for blood and/or other biological analysis that may avoid the use of a blood draw.

### SUMMARY

Self-calibrating adaptive optical systems, devices, and methods for biological analyte measurements are provided in accordance with various embodiments. For example, some embodiments include a method that may include emitting light from a laser. The light from the laser may be filtered utilizing one or more short pass filters. The filtered light from the laser may be focused onto one or more objects. Light may scatter from the one or more objects. The scattered light from the one or more objects may be filtered utilizing a long pass filter. The scattered light from the one or more objects may be collected into a spectrometer aperture. The scattered light from the one or more objects may pass through a spectrometer to spatially separate the spectral components of the scattered light into a scattered spectra; the scattered spectra may be filtered and detected on one or more optical sensors to form a detected spectra. The detected spectra may be digitized and processed to deliver a Raman spectra to a processor for analysis.

Some embodiments of the method include beam steering the light from the laser utilizing one or more first spatial light modulators. Utilizing one or more first spatial light modulators may include utilizing one or more MEMs mirrors. Some embodiments further include beam shaping the scattered light from the one or more objects utilizing one or more second spatial light modulators.

Some embodiments of the method include placing one or more body parts as the one or more objects and collecting the Raman spectra from the one or more body parts. Some embodiments include moving the one or more body parts in response to an indicator. The indicator may be proportional to one or more analyte measurements calculated from the Raman spectra. The one or more analyte measurements may include a hemoglobin measurement. Some embodiments include utilizing a targeting image sensor and an imaging system such that an image of the one or more body parts forms on the targeting image sensor and an image from the image sensor is communicated to the processor to control a MEMS mirror; indicator may be a measured overlap of the illumination beam with a vein of the human hand as measured using the targeting image sensor. Some embodiments include measuring an analyte using the Raman spectra when one or more analyte measurements fall within a specific range.

Some embodiments include processing the Raman spectra that may include: removing background scattered light from the Raman spectra to produce a bias-free spectra; calculating two or more coarse analyte measurements as an inner product of the bias-free spectra with two or more reference analyte spectra; and calculating two or more refined analyte measurements by compensating for multiple analyte inner products. Compensating for the multiple analyte inner products may include: loading the two or more coarse analyte measurements into a vector; and multiplying the vector by an inverse of a matrix of inner products from a library of spectra within the two or more reference analyte spectra.

Some embodiments of the method include determining if one or more of the refined analyte measurements are sufficient. The one or more values of the one or more refined analyte measurements may be utilized to control a collection of the Raman spectra. Calculating the two or more coarse analyte measurements may include: modeling the Raman spectra as a weighted sum of a reference spectra; and estimating a plurality of weights that deliver a minimum mean square estimate of the bias-free spectra.

Some embodiments of the method include processing the Raman spectra that may include processing two or more digital spectra to produce two or more sets of refined analyte measurements. Some embodiments include: calculating one or more amplitude closure values from the two or more sets of refined analyte measurements, and calculating one or more errors in one or more amplitude closure values by multiplying the amplitude closure values by a pseudoinverse of an amplitude closure matrix. Some embodiments correct the two or more sets of refined analyte measurements by removing the errors. This result may be deemed one example of a self-calibrated analyte measurement.

Some embodiments include a system that may include a light source, such as a laser. The light from the laser may diverge from a first focus to form a diverging laser light. A first concave mirror may be positioned one focal length from the first focus such that the diverging laser light may be reflected from the first concave mirror as a first collimated light beam. A short pass filter may be positioned in at least the diverging laser light or the first collimated light beam. One or more substrates may be positioned one focal length from the first concave mirror; the one or more substrates may include one or more substrate apertures or openings such that the first collimated light beam passes through at least one of the one or more substrate apertures. A second concave mirror may be positioned two focal lengths from the first concave mirror such that the second concave mirror focuses the first collimated light beam to a second focus a first diverging light beam. A MEMs mirror may be positioned at the second focus; the MEMs mirror may reflect the first diverging light beam back to the second concave mirror such that the second concave mirror collimates the diverging light beam as a second collimated light beam. In some embodiments the spatial light modulator is utilized instead of a MEMs mirror. A first reflector may be positioned one focal length from the second concave mirror such that the first reflector reflects the second collimated light beam off of the second concave mirror to a third focus within at least one of the one or more substrate apertures to form a second diverging light beam. A third concave mirror may be positioned one focal length from the third focus; the third concave mirror may collimate light from the third focus into a third collimated beam and directs the third collimated beam to an object plane.

Some embodiments include a variation on this system where the laser is configured such that light from the laser is collimated at a first focus to form a collimated laser light. The first concave mirror may be positioned one focal length from the first focus such that the collimated laser light is focused by the first concave mirror to a second focus as a first converging light beam. The short pass filter may be positioned in at least the collimated laser light or the first converging light beam. The one or more substrates may be positioned one focal length from the first concave mirror; the one or more substrates may include one or more substrate apertures such that the first converging light beam passes through at least one of the one or more substrate apertures. The second concave mirror may be positioned two focal lengths from the first concave mirror such that the second concave mirror collimates the first converging light beam to a third focus as a second collimated light beam. The MEMs mirror may be positioned at the third focus; the MEMs mirror may reflect the second collimated light beam back to the second concave mirror such that the second concave mirror focuses the second collimated light beam as a second converging light beam. The first reflector may be positioned one focal length from the second concave mirror such that the first reflector reflects the second converging light beam off of the second concave mirror to a fourth focus within at least one of the one or more substrate apertures to form a third collimated light beam. The third concave mirror may be positioned one focal length from the fourth focus, wherein the third concave mirror focuses light from the third collimated light beam into a third converging beam and directs the third converging beam to an object plane.

The systems may be configured such that one or more positions of the object scatter light back to the third concave mirror or a fourth concave mirror as one or more diverging object beams such that the one or more first diverging object beams are collimated by the third concave mirror or the fourth concave mirror to form one or more first collimated object beams that pass through at least one of the one or more substrate apertures and reflect off the second concave mirror or a fifth concave mirror to a first object beam focus to form one or more first converging object beams. A second reflector may be positioned one focal length from the second concave mirror or the fifth concave mirror, where at least a portion of the second reflector is positioned at the first object beam focus such that the second reflector reflects the one or more first converging object beams from the first object beam focus as one or more second diverging object beams to the second concave mirror or the fifth concave mirror and the second concave mirror or the fifth concave mirror collimates the one or more second diverging object beams to produce one or more second collimated object beams. A third reflector may be positioned one focal length from the second concave mirror or the fifth concave mirror; the third reflector may reflect the one or more second collimated object beams back at the second concave mirror or the fifth concave mirror such that the one or more second collimated object beams are focused to a second object beam focus within at least one of the one or more substrate apertures to form one or more third diverging object beams that are directed toward the first concave mirror or a sixth concave mirror such that the first concave mirror or the sixth concave mirror collimates the one or more third diverging object beams to form one or more third collimated object beams. A first long pass filter may be positioned within the one or more third collimated object beams. A diffractor may be positioned one focal length from the first concave mirror or the sixth concave mirror and may intersect a portion of the one or more third collimated object beams such that the one or more third collimated object beams diffracts according to a plurality of wavelengths of the one or more third collimated object beams and the plurality of wavelengths of the one or more third collimated object beams are directed at the first concave mirror or the sixth concave mirror such that a diffracted beam at each wavelength from the plurality of wavelengths is focused to a different position of the first concave mirror or the sixth concave mirror to form a spectra of focused beams. A second long pass filter may be positioned in the spectra of focused beams. A detector array may be positioned in the spectra of focused beams such that the spectra of focused beams are detected, sampled, and communicated to a processor as a detected spectra.

In some embodiments of the systems, the first reflector is a long pass filter positioned in front of a targeting image sensor such that the long pass filter reflects light from the laser while transmitting light at longer wavelengths than the laser to the targeting image sensor. The targeting image sensor may be positioned in an image plane of the object and may collect one or more images using light from a second illumination source.

In some embodiments of the system, the second reflector is a spatial light modulator. In some embodiments, the laser, the short pass filter, the first long pass filter, the diffractor, the second long pass filter, and the detector array are mounted to a first substrate from the one or more substrates. In some embodiments, the MEMS mirror, the first reflector, the second reflector, and the third reflector are mounted on a second substrate from the one or more substrates.

In some embodiments, the laser and the short pass filter are mounted on the substrate. In some embodiments, the first long pass filter, the diffractor, the second long pass filter, and the detector array are also mounted on that substrate. In some embodiments, the first long pass filter, the diffractor, the second long pass filter, and the detector array are mounted on another substrate.

In some embodiments, the first spatial light modulator and the first reflector are mounted to the same substrate. In some embodiments, the second reflector (or second spatial light modulator) and the third reflector are mounted substrate on that same substrate. In some embodiments, the second reflector (or second spatial light modulator) and the third reflector are mounted on another substrate.

In some embodiments, a volume separates at least the first concave mirror, the second concave mirror, or the third concave mirror from a plane of the second substrate is filled with a material transparent at a wavelength of the laser. The material filling the volume may be safe for use in-vivo with biological systems.

Some embodiments of the systems include: a power supply; one or more electrical controls; one or more temperature sensors; and/or a processor such that: a current driving the laser is configured to be controlled; a temperature of the laser is configured to be controlled; the MEMS mirror is configured to be tilted along one or more axes; the targeting image sensor is configured to be powered on; the targeting image sensor is configured to be configurable; one or more settings of the targeting image sensor are configured to be communicated to at least the processor or the electrical controls; the image from the targeting image sensor is configured to be communicated to the processor; the spatial light modulator is configured to be powered on; the spatial light modulator is configured to be configurable; one or more settings of the spatial light modulator are configured to be communicated to at least the processor or the electrical controls; and a value of one or more temperature sensors is configured to be communicated to at least the processor or the electrical controls.

Some embodiments of the systems include a biologically inert and optically transparent shell that houses the device such that the device is configured to be implanted into a person. The device may be configured to be placed under the skin of a person.

In some embodiments of the systems, the laser includes an array of lasers and the MEMS mirror is a spatial light modulator. The array of lasers may include a VCSEL array.

In some embodiments, the systems are manufactured where one step of the manufacturing is the dicing of a multi-layered wafer comprising: a spectrometer integrated optic layer; a spectrometer substrate layer; a steering substrate layer; and a steering integrated optic layer.

In some embodiments, the wavelength of the laser is stabilized to less than 2 nm. The short pass filter may include a passband aligned to the wavelength of the laser that rejects light more than 20 nm longer than the wavelength of the stabilized laser by at least 30 dB; the first long pass filter may reject light at the wavelength of the laser by at least 30 dB and passes light more than 20 nm from the wavelength of the laser. The second long pass filter may reject light at the wavelength of the laser by 30 dB and may pass light more than 20 nm from the wavelength of the laser.

Some embodiments include a method that include: emitting light from a source; directing, filtering, and focusing the light onto one or more objects; scattering light from the one or more objects; collecting and filtering, the scattered light from the one or more objects into a spectrometer aperture; passing the scattered light that is collected and filtered through a spectrometer to spatially separate the spectral components of the scattered light into a scattered spectra where the scattered spectra is filtered and detected on one or more optical sensors to form a detected spectra; and amplifying, digitizing, processing, and communicating the detected spectra to deliver a Raman spectra to a processor for analysis.

In some embodiments of the method, directing the light onto the one or more objects utilizes one or more spatial light modulators. Some embodiments include positioning the one or more spatial light modulators in a Fourier plane of the one or more objects within a Fourier optical system such that a beam angle produced by the one or more spatial light modulators shifts one or more positions at which one or more beams is focused on the one or more objects. In some embodiments, the source includes a multi-mode source and the one or more spatial light modulators are placed in a Fourier plane of the one or more objects within a Fourier optical system such that a beam angle produced by each of the one or more spatial light modulators shifts one or more positions at which two or more beams are focused on the one or more objects. In some embodiments, the one or more spatial light modulators include one or more MEMs mirrors.

Some embodiments of the method include forming an image of the one or more objects on a targeting imaging sensor. Some embodiments include controlling the one or more spatial light modulators utilizing the image of the one or more objects on the targeting image sensor.

In some embodiments of the method, the Fourier optical system includes a concave mirror that includes a conic curvature. In some embodiments, the one or more spatial light modulator and the targeting image sensor are mounted on a planar surface. In some embodiments, the one or more optical sensors and the diffractor of the spectrometer are co-planar and/or on another planar surface.

Some embodiments of the method include utilizing an analyte measurement method that processes the digitized spectra to deliver one or more analyte measurements. The one or more spatial light modulators may be controlled to maximize one or more analyte measurements.

Some embodiments of the method include beam shaping the scattered light utilizing a spatial light modulator. The spatial light modulator may be positioned in the Fourier plane of the one or more objects such that each spectral frequency of the spatial light modulator diffracts light to a resolvable position in a plane of the spectrometer aperture of the spectrometer. Some embodiments include utilizing an analyte measurement method that processes the digitized spectra to deliver one or more analyte measurements, wherein the spatial light modulator is controlled to maximize one or more analyte measurements. Some embodiments of the method include beam shaping the scattered light utilizing an array of two or more MEMS mirrors.

In some embodiments, a Fourier optical system is constructed utilizing a concave mirror that is hyperbolic. In some embodiments, the spatial light modulator, the MEMS mirror, and the targeting image sensor are co-planar. In some embodiments, the optical sensor and the diffractor of the spectrometer are co-planar with the source.

Some embodiments of the method include: placing a human hand as the one or more objects; and collecting the Raman spectra from the human hand. Some embodiments include moving the human hand in response to an indicator. The indicator may be proportional to the one or more analyte measurements. The one or more analyte measurements may include a hemoglobin measurement. Some embodiments include utilizing a targeting image sensor and an imaging system such that an image of the human hand forms on the targeting image sensor and an image from the image sensor is communicated to the processor to control a MEMS mirror; the indicator may be a measured overlap of the illumination beam with a vein of the human hand as measured using the targeting image sensor.

Some embodiments of the method include processing the Raman spectra that includes: removing background scattered light from the Raman spectra to produce a bias-free spectra; calculating two or more coarse analyte measurements as an inner product of the bias-free spectra with two or more reference analyte spectra; and calculating two or more refined analyte measurements by compensating for multiple analyte inner products. Compensating for the multiple analyte inner products may include: loading the two or more coarse analyte measurements into a vector; and multiplying the vector by an inverse of a reference inner product matrix within the two or more reference analyte spectra. Some embodiments include analyzing the Raman spectra for one or more analytes to determine if one or more values of the one or more analytes are sufficient. The one or more values of the one or more analytes may be utilized to control a collection of the Raman spectra. Calculating the two or more coarse analyte measurements may include: modeling the Raman spectra as a weighted sum of a reference spectra; and estimating multiple weights that deliver a minimum mean square estimate of the bias-free spectra.

In some embodiments of the method, processing the Raman spectra includes processing two or more digital spectra to produce two or more sets of refined analyte measurements. Some embodiments include: calculating one or more amplitude closure values from the two or more sets of refined analyte measurements, and calculating one or more errors in one or more amplitude closure values by multiplying the amplitude closure values by a pseudoinverse of an amplitude closure matrix. Some embodiments include correcting the two or more sets of refined analyte measurements by removing the one or more errors.

Some embodiments include devices, systems, and/or methods as described in the specification and/or shown in the figures.

The foregoing has outlined rather broadly the features and technical advantages of embodiments according to the disclosure in order that the detailed description that follows may be better understood. Additional features and advantages will be described hereinafter. The conception and specific embodiments disclosed may be readily utilized as a basis for modifying or designing other structures for carrying out the same purposes of the present disclosure. Such equivalent constructions do not depart from the spirit and scope of the appended claims. Features which are believed to be characteristic of the concepts disclosed herein, both as to their organization and method of operation, together with associated advantages will be better understood from the following description when considered in connection with the accompanying figures. Each of the figures is provided for the purpose of illustration and description only, and not as a definition of the limits of the claims.

### BRIEF DESCRIPTION OF THE DRAWINGS

A further understanding of the nature and advantages of different embodiments may be realized by reference to the following drawings. In the appended figures, similar components or features may have the same name and/or same reference number with respect to the last two digits of a three digit number. Further, various components of the same type may be distinguished by following the reference label by a dash and a second label that distinguishes among the similar components. If only the first reference label with respect to the three digit number is used in the specification, the description is applicable to any one of the similar components having the same first reference label irrespective of the second reference label.

FIG. 1 shows a system in accordance with various embodiments.

FIG. 2A and FIG. 2B show systems in accordance with various embodiments.

FIG. 3A, FIG. 3B, and FIG. 3C show a system in accordance with various embodiments.

FIG. 4A, FIG. 4B, FIG. 4C, FIG. 4D, FIG. 4E, FIG. 4F, FIG. 4G, and FIG. 4H show systems in accordance with various embodiments.

FIG. 5 shows a system in accordance with various embodiments.

FIG. 6 shows a system in accordance with various embodiments.

FIG. 7 shows a flow diagram of a method in accordance with various embodiments.

FIG. 8A shows a flow diagram of a method in accordance with various embodiments.

FIG. 8B shows an infrared image and associated graph of one image row in accordance with various embodiments.

FIG. 9 shows a flow diagram of a method in accordance with various embodiments.

FIG. 10 shows a flow diagram of a method in accordance with various embodiments.

### DETAILED DESCRIPTION

This description provides embodiments, and is not intended to limit the scope, applicability, or configuration of the disclosure. Rather, the ensuing description will provide those skilled in the art with an enabling description for implementing embodiments of the disclosure. Various changes may be made in the function and arrangement of elements.

Thus, various embodiments may omit, substitute, or add various procedures or components as appropriate. For instance, it should be appreciated that the methods may be performed in an order different than that described, and that various stages may be added, omitted, or combined. Also, aspects and elements described with respect to certain embodiments may be combined in various other embodiments. It should also be appreciated that the following systems, devices, and methods may individually or collectively be components of a larger system, wherein other procedures may take precedence over or otherwise modify their application.

Self-calibrating adaptive optical systems, devices, and methods for biological analyte measurements are provided in accordance with various embodiments. The systems, devices, and/or methods may use adaptive optics to relay light into and out of diffuse environments. The adaptive optics may enable 20-40 dB of suppression of the signal over that of a typical optical microscope. These systems, devices, and/or methods may be paired with other analysis sensors in order to enhance the signals of interest. Some embodiments are focused on the biological analysis space.

Various embodiments include optical analysis devices and photonic integrated circuit (PIC) systems and/or freespace optical devices to deliver analysis of biological signatures are provided. These systems and/or devices may be sensitive when the sample is prepped. Blood, for example, may be sorted for proper optical analysis. For some analysis, however, a processor may be able to use the same technology to produce in-situ analysis of the blood—provided the optical signal may be focused onto the blood vessel. For this application, various embodiments enable biological analysis of the blood to be performed through the skin. Such a capability may enable the delivery of some forms of blood analysis without involving a blood draw. By delivering blood analysis without a blood draw, patients may be tested in a wide variety of environments—including public kiosks, automated pharmacies, cellphone, office health stations, in their own home, and even from their wearable device. Moreover, because testing may be easier, the number of times the tests may be performed can be dramatically increased. Tests may move from annually to monthly, weekly, daily, hourly, or continuously. Ever-present sensing can be achieved if the blood draw may be removed.

Various embodiments may utilize Raman spectroscopy, a method by which one can study the chemical composition of a substance in a contact-free method. When illuminated by a laser beam, molecules scatter a small fraction of that laser beam with a generally lower frequency. This frequency shift in the scattered light is generally called the Stokes shift, and it is generally caused by the excitation of a molecular vibration or bending moment in the molecule. When the molecule absorbs the energy of the moment, less energy may be scattered, which is exhibited by the scattering of light at a generally lower frequency. Because each molecule has a unique combination of vibration and bending modes, the collection of moments is unique and so the spectra of scattered light is unique. Raman spectroscopy generally examines the scattered light as a method to detect, identify, and measure molecules by studying the spectra of scattered light. The lower frequencies of Stokes shifted light correspond to longer wavelengths, so for Stokes shifted light, Raman spectroscopy systems may be configured to study wavelengths longer than the excitation beam. Raman spectroscopy systems may also study anti-Stokes shifts—wherein the wavelengths of light are shorter than the excitation beam.

The various systems, devices, and methods provided in accordance with various embodiments typically operate by illuminating the object, collecting light scattered from the object, spectrally filtering the scattered light, forming the spectra of the scattered light, analyzing the spectra of the scattered light to measure the analytes, and reporting the analyte measurements to a user or other tool. Some embodiments provide these tools and techniques in order to deliver analyte measurements of the biological processes within a person or living system where the analyte measurements may provide a useful indicator of the health or wellness of the person or living system. Some embodiments include systems and/or devices with innovative optical configurations that may enable high performance, compact, and low cost systems and/or devices. The systems and/or devices provided in accordance with various embodiments may include innovative structures, fabrication methods, and form factors that may enable compact devices. Methods are provided in accordance with various embodiments that may deliver higher sensitivity and a larger tolerance to stray light than may be produced using other methods.

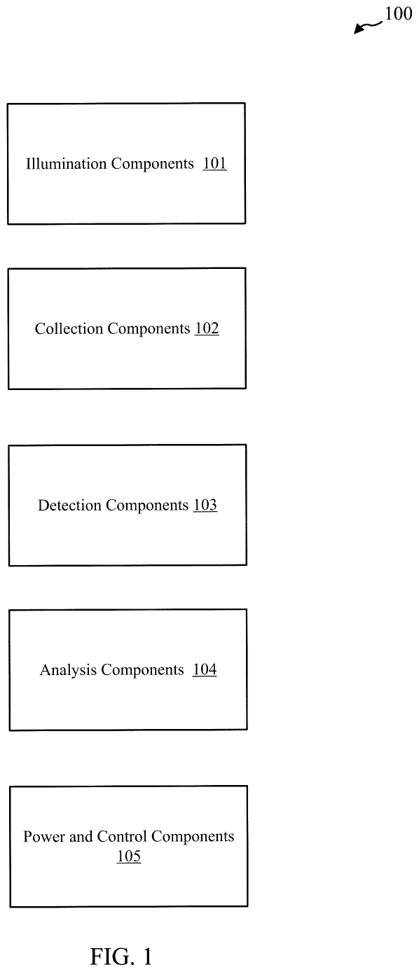

FIG. 1 provides an example of a general system 100 in accordance with various embodiments. System 100 may utilize a wide variety of components that may be gathered into various groups, which sometimes may be referred to as devices and/or modules. For example, system 100 may include illumination components 101. The illumination components 101 may include, but are not limited to, light sources, lenses, mirrors, filters, and/or spatial light modulators. The illumination components 101 may be referred to also as source-side components. System 100 may include various collection components 102, which may be referred to as specimen or object-side components. The collection components 102 may include, but are not limited to, light sources, lenses, mirrors, filters, and/or spatial light modulators. In some embodiments, the illumination components 101 and the collection components 102 may share some of their components. System 100 may include various detection components 103, which may include, but are not limited to, lenses, mirrors, diffractors, filters, and/or detection arrays. One or more of these components may form a spectrometer. System 100 may include various analysis components 104, which may include, but are not limited to, processors. System 100 may include a variety of other components that may generally be referred to as power and control components 105. This may include, but is not limited to, power supplies, electrical controls, sensors, and additional illumination sources.

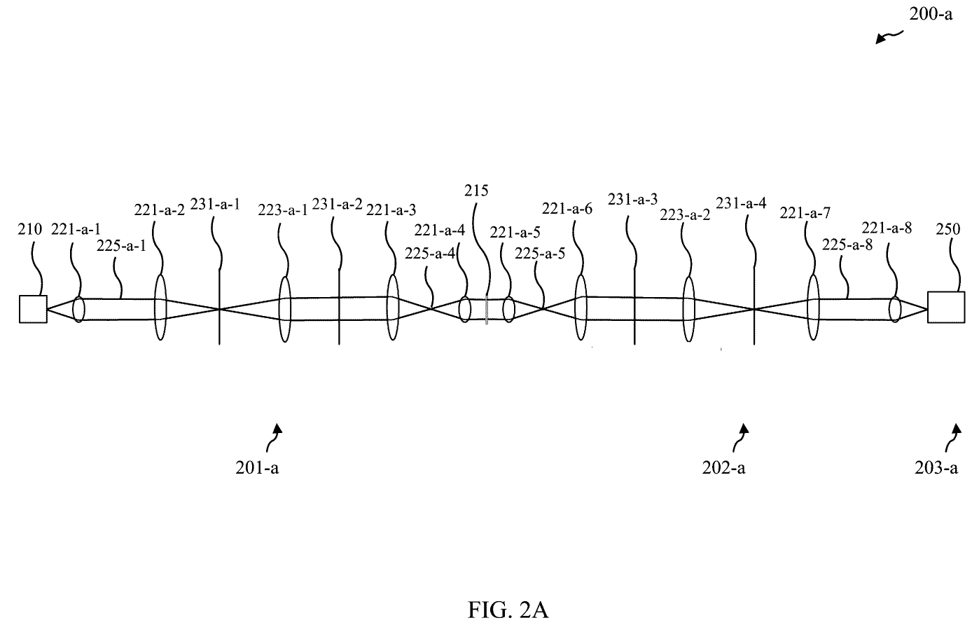

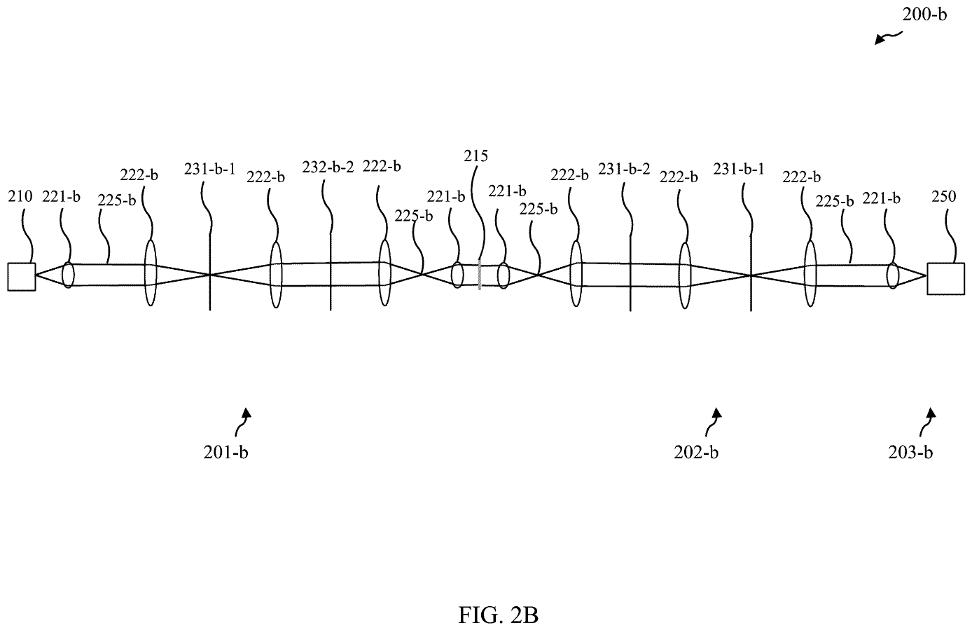

Turning now to FIG. 2A and FIG. 2B, two examples of systems 200-a and 200-b are provided in accordance with various embodiments. These examples may be referred to as spatial light modulator adaptive optic systems for adaptive illumination. Systems 200-a and 200-b may be examples of system 100 of FIG. 1.

Systems 200-a and 200-b may provide examples of freespace optical systems that may illuminate a specimen 215 including the use of one or more source-side 4-F imaging modules, 2-F Fourier transform module, and one or more specimen-side 2-F imaging modules.

FIG. 2A provides an example of a lens-based version of system 200-a, which may include lens 221-a-1 and lens 221-a-2 that may form the source-side 4-F imaging module. Fourier transform lens 223-a-1 may form parts of the 2F Fourier transform module. Lens 221-a-3 and lens 221-a-4 may form the specimen-side 4-F imaging module. FIG. 2B provides an example of a mixed lens and mirror-based system 200-b in general. System 200-b is shown from a propagation path perspective. Lens 221-b and/or concave mirror 222-b may form parts of the source-side 4-F imaging module, the 2F Fourier transform module, and the specimen-side 4-imaging modules. System 200-a may generally be referred to as a transmissive system while system 200-b may generally be referred to as a reflective system.

A source 210 may be included at the input of the source-side 4-F imaging module. In some embodiments, the source 210 may be emitted by a PIC. In other embodiments, the source 210 may be an edge emitting laser, a VCSEL, a single-mode optical fiber emitting light, a multi-mode optical fiber emitting light, and/or a single mode waveguide emitting light. In some embodiments, the source 210 is polarized.

A spatial light modulator 231-a-1/231-b-1 may be included at an output of the source-side 4-F imaging components; spatial light modulator 231-a-1/231-b-1 may be referred to as a first spatial light modulator. In some embodiments, the spatial light modulator 231-a-1 is transmissive and may be constructed as a transmissive liquid crystal device, an array of electro-optic modulators, a tunable wedge, an acousto-optic deflector, a photo-refractive crystal written by additional optical beams, a spectral hole burning crystal written by additional optical beams, and/or other transmissive modulator systems. In some embodiments, spatial light modulator 231-b-1 is a reflective spatial modulator, including, but not limited to, a LCOS chip. In such an embodiment, the optical components of a device may generally be reflective where the LCOS chip is used, such as with FIG. 2B. In some embodiments, the output of source 210 is polarized using a polarizing element such as a prism or an integrated polarizer.

The light emitted by the source 210 may be imaged onto the spatial light modulator 231-a-1/231-b-1 through the source-side 4-F imaging module. At the spatial light modulator 231-a-1/231-b-1, the phase and amplitude of the light may be modulated. In the case of an LCOS chip, the phase of the light may be modulated for one particular polarization. The phase modulation may be a superposition of many phase ramps. The light modulated by the first spatial light modulator 231-a-1/231-b-1 may then enter the 2F Fourier transform module.

One may define a 2F Fourier transform module as a module in which the transverse electric field at the output of the system may be the Fourier transform of the electric field at the input to the system. In some embodiments, the output may be distorted or may experience aberrations that may deviate from the perfect Fourier transform. Such effects may limit the performance and may generally be tolerated.

The light at the output of the 2F Fourier transform module may approximate the modulation of the first spatial light modulator 231-a-1/231-b-1, convolved by the Fourier transform of the input beam. One may recognize that a 2F Fourier transform may be replaced by two lenses separated by a single focal length; in this variation, the Fourier transform would generally be telecentric.

In some embodiments, the Fourier transform mapping onto the transverse plane is represented as an F-sine-theta relationship. In some embodiments, the Fourier transform is represented as an F-tan-theta relationship. In embodiments wherein the mapping does not exactly map a Fourier transform, the modulation elements of the array may be designed to accommodate the new mapping.

The light at the output of the 2F Fourier transform module may then be modulated by a spatial light modulator 231-a-2/231-b-2, which may be referred to as a second spatial light modulator and passed into the 2F Fourier transform module.

The output from the 2F Fourier transform module may then be passed into the specimen-side 4-F imaging module, and the image of the light may fall onto the specimen 215. In some embodiments, the light focuses in a generally planar configuration. In some embodiments, the light at different positions is focused into generally non-planar positions. In some embodiments, the focus may vary as a monotonic function of the radial distance from an optical axis of the system 200-a/200-b. In some embodiments, the focus may vary according to the desired image position. In some embodiments, the variation in focus is imparted by the modulation of first spatial light modulator 231-a-1/231-b-1 and/or the second spatial light modulator 231-a-2/231-b-2. In some embodiments, a microscope objective is included in the 4-F imaging module. In some embodiments, the working distance of the microscope objective is generally long. In some embodiments, the microscope objective is generally telecentric.

Some embodiments are configured to provide an adaptive optic method for the collection of light from the specimen 215 and formation onto one or more detector ports 250 at the output of the system 200-a/200-b. For example, systems 200-a/200-b may include a specimen-side 4-F imaging module, a 2F Fourier transform imaging module, and a detector-side 4-F imaging module.

FIG. 2A again provides generally a lens-based version where lens 221-a-5 and lens 221-a-6 may form parts of the specimen-side 4-F imaging module. Fourier transform lens 223-a-2 may form parts of the 2-F Fourier imaging transform module. Lens 221-a-7 and lens 221-a-8 may form parts of the detector-side 4-F imaging module. For FIG. 2B, lens 221-b and/or concave mirror 222-b may form parts of the source-side 4-F imaging module, the 2F Fourier transform module, and/or the specimen-side 4-imaging modules.

Light transmitted through the specimen 215 may be reflected or scattered at many different angles and many different positions. This light may be collected through the specimen side 4-F imaging module and imaged onto spatial light modulator 231-a-3/231-b-2. In some embodiments, a microscope objective is included in the specimen side 4-F imaging module. In some embodiments, a working distance of the microscope objective is generally long. In some embodiments, the microscope objective is generally telecentric. Light from the specimen 215 may then be modulated by spatial light modulator 231-a-3/231-b-2. In some embodiments, the modulation is chosen to maximize the light falling onto one of the detector ports 250. In some embodiments, this is done such that the light on the detector port 250 may fall generally in an imaging configuration.

One benefit of various embodiments is that additional signal may be captured when light from additional locations and angles are additionally focused onto the detector port 250. In some embodiments, the configuration generally deviates from an imaging configuration. Methods for optimizing the spatial light modulators are generally described later in this detailed description.

When modulated light in FIG. 2A passes through spatial light modulator 231-a-3, it may pass through the Fourier transform lens 223-a-2 and may fall onto spatial light modulator 231-a-4; for FIG. 2B, modulated light from spatial light modulator 231-b-2 may reflect off concave mirror 222-b and may fall onto spatial light modulator 231-b-1. In some embodiments where the detector port 250 has a phase sensitive collection structure, spatial light modulators 231 may be used to maximize the light coupled into the collection structure. In some embodiments, this phase sensitive collection structure includes an optical fiber. In some embodiments, the phase sensitive collection structure is a waveguide, a PIC, and/or a coherent detector with a coherent light beam. For example, spatial light transmitter 231-a-4/231-b-1 may then be optimized such that the phase of the output modulation may be compensated for maximal coupling. Methods of such optimization are described later in this detailed description.

Light from spatial light modulator 231-a-4/231-b-1 may be relayed onto detector port 250 using the 4-F imaging module. In some embodiments, the light of the output for spatial light modulator 231-a-4/231-b-1 is generally imaged onto the detector port 250 utilizing lens 221-a-7 and 221-a-8 (for FIG. 2A) or concave mirror 222-b and lens 221-b (for FIG. 2B). In some embodiments, the light on the detector port 250 may deviate from an image of the specimen 215.

One may also note that several Fourier planes 225-a-1, 225-a-4, 225-a-5, and 225-a-8 may be referred to in FIG. 2A. Fourier plane 225-b in FIG. 2B may be called out in several locations.

Light at the detector port 250 may be coupled onto a myriad of optical analysis tools. In some embodiments, the optical analysis tool includes a fiber coupled OCT sensor. In some embodiments, the optical analysis tool includes a PIC configured to detect glucose. In some embodiments, the PIC is configured to detect oxidized hemoglobin. Other embodiments include analysis for progesterone, estrogen, testosterone, lactic acid, and/or many other detectable compounds. Other embodiments include analysis for specific chemical markers. The point of various embodiments is to remain compatible with the technologies developed around the world. Some embodiments provide methods to move the focus of light for analysis deep into a scattering structure.

In various embodiments, such scattering structure may include the skin of the finger, the back of the hand, a turbulent fluid, a turbulent atmosphere, etc. In some embodiments, the scattering structure may be non-existent such that there is no need for spatial light modulation.

In general, components shown to the left of specimen 215 in system 200-a/200-b may be referred to as illumination components 201-a/201-b. In general, components shown to the right of specimen 215 may be referred to as collection components 202-a/202-b, until detector port 250 is reached, which may represent the beginning of the detection components 203-a/203-b. In some embodiments, illumination components 201-a/201-b include one or more short pass filters. In some embodiments, collection components 202-a

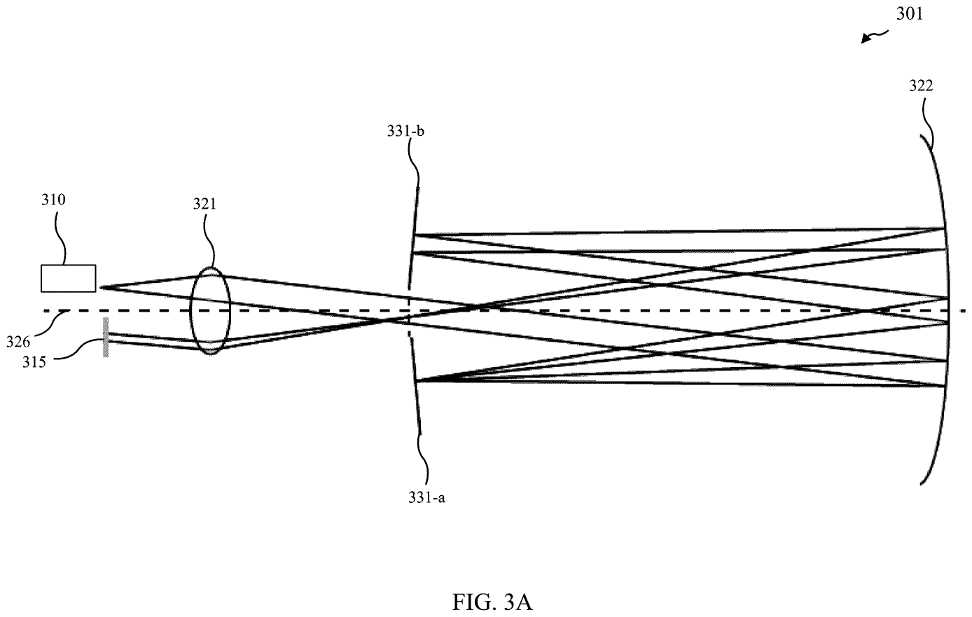

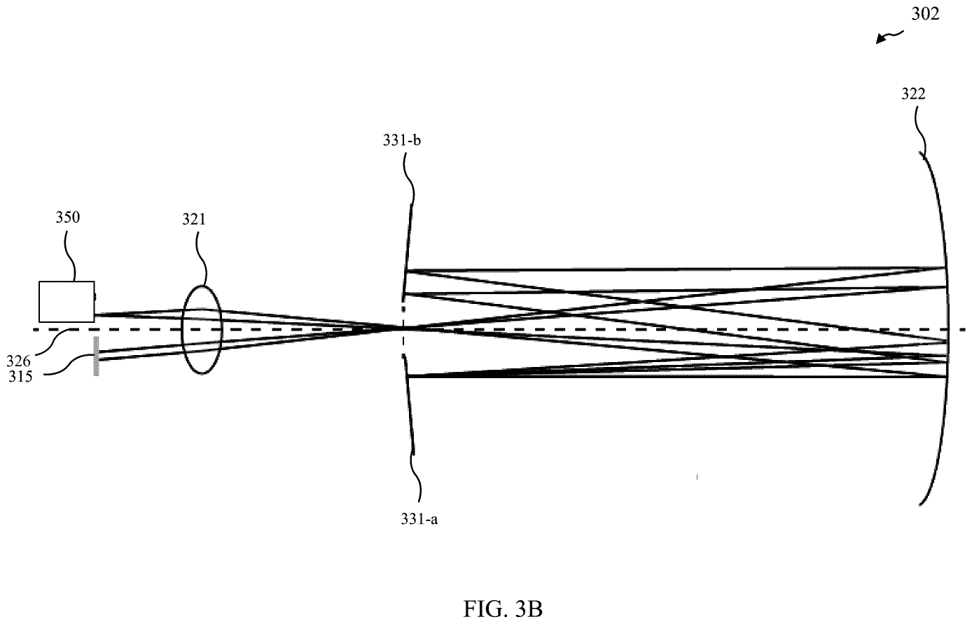

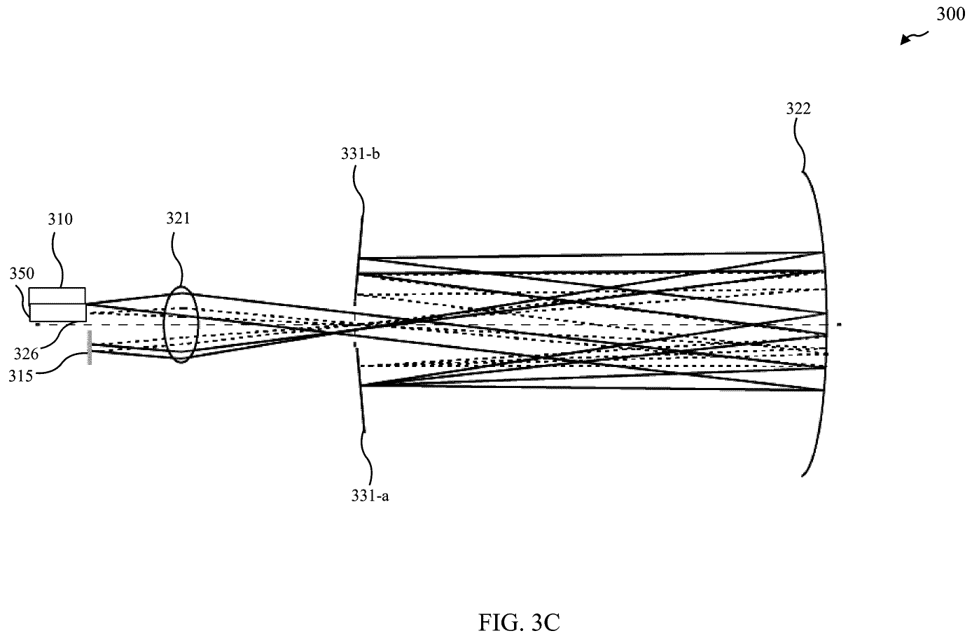

Turning now to FIG. 3A, FIG. 3B, and FIG. 3C, examples of devices 301 and 302 along with system 300 are provided in accordance with various embodiments. Devices 301 and 302 and system 300 may provide examples of reflective adaptive optic imaging devices and/or systems. These devices and system may provide various advantages over other existing technologies generally available. These devices and system may help reduce losses and take advantage of low cost reflective spatial light modulators, including LCOS technology. Device 301 may be an example of illumination components 101 of FIG. 1. In some embodiments, device 301 includes one or more short pass filters. Device 301 may be an example of collection components 102 of FIG. 1. In some embodiments, device 302 includes one or more long pass filters. The collection device 302 may also be referred to as a reflection device. System 300 provides an example where devices 301 and 302 may utilize the same components to form a system with both illumination and collection features. System 300 may include one or more short pass filters with respect to its illumination components and one or more long pass filters with respect to its collection components.

Device 301 of FIG. 3A includes a source-side 4-F imaging module, a 2-F Fourier transform module, and a specimen-side 4-F imaging module. In this configuration, lens 321 and the concave mirror 322 may form the source-side 4-F imaging module that may image a source 310 onto a first spatial light modulator 331-a, which may be a reflective spatial light modulator. In some embodiments, the spatial light modulator 331-a is an array of modulators such as the LCOS devices included in displays.

In some embodiments, the 4-F imaging module is constructed using a first Fourier transform lens followed by convex reflecting mirror. One extension of this embodiment would use a generally hyperbolic mirror. One extension of this embodiment would include a generally elliptic mirror.

In some embodiments, light from first spatial light modulator 331-a, which may be reflective, passes through a 2F Fourier transform module by reflecting off the concave mirror 322 to land on a second spatial light modulator 331-b, which may be placed generally in the Fourier transform location of second spatial light modulator 331-b. As with a device based on lenses, the Fourier transform may deviate. It may result in an F-tan-theta or an F-sin-theta configuration. It may be generally telecentric. The spatial light modulator technology used at the second spatial light modulator 331-b may differ substantially from the technology used at the first spatial light modulator 331-a. The spatial light modulator used at the second spatial light modulator 331-b may be a spatially distinct portion of the spatial light modulator used at the first spatial light modulator 331-a. In some embodiments, a slight tilt between the input and output reflection from the convex reflecting mirror 322 may separate the beams.

In some embodiments, light reflected from the second spatial light modulator 331-b then passes through a 4-F imaging module that includes the concave mirror 322 and lens 321 such that the image of light from second spatial light modulator 331-b falls onto the specimen 315.

When light is reflected from the specimen 315 illuminated by device 301 of FIG. 3A, it may be collected using similar optics as shown in FIG. 3B. This may generally provide for a reflective adaptive optic light collection module. In some embodiments, the exact same optics are used, as may be shown in FIG. 3C. For example, light from the specimen 315 may be passed back through the 4F imaging module that may include the lens 321 and the concave mirror 322 to land in an imaging configuration at the second spatial light modulator 331-b, which may be reflective. The second spatial light modulator 331-b may modulate the reflected light. The reflected light may then pass through the 2-F Fourier transform module that may include the concave mirror 322 such that the light lands on the first spatial light modulator 331-a. The first spatial light modulator 331-a may modulate the light and then pass it into the 4-F imaging system that may include the concave mirror 322 and lens 321 such that the light may be placed onto one or more detector ports 350. An optical axis 326 may also be referred to in the figures.

FIG. 3C shows the transmitted and reflected light from the specimen 315 overlayed in the same optical device or system. In some embodiments, the reflected light for one illumination direction is generally along a single direction. In some embodiments, the reflection is more diffuse and the reflected light may travel generally along multiple directions. Several innovative strategies discussed below discuss multiple directions of illumination.

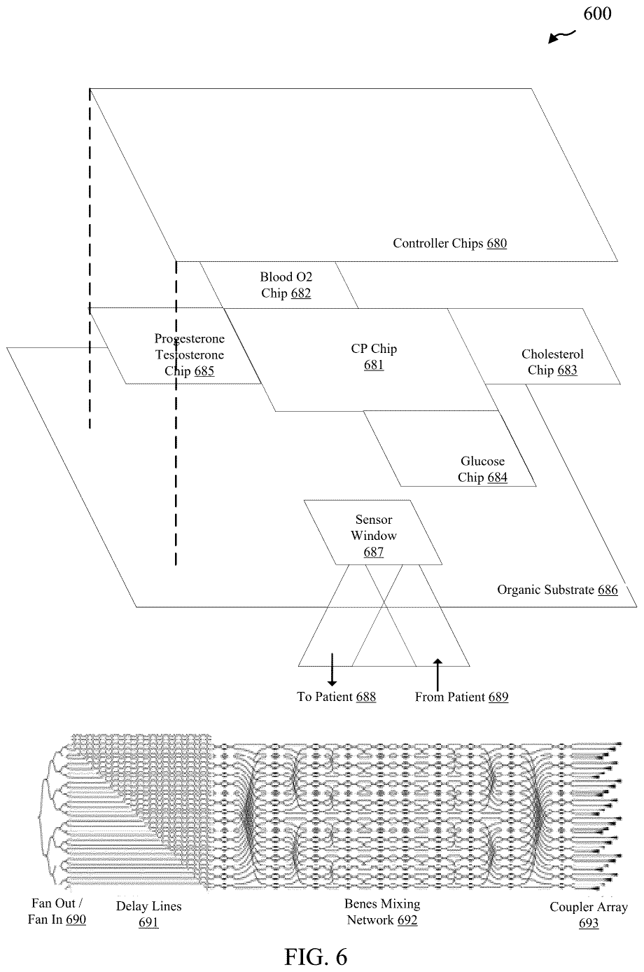

The first spatial light modulator 331-a and the second spatial light modulator 331-b of FIG. 3A, FIG. 3B, and/or FIG. 3C provide various functions. These functions may also be generally applicable to other embodiments, such as those shown with regard to the spatial light modulators of FIGS. 2, FIG. 4, FIG. 5, and/or FIG. 6. These functions are generally discussed with respect to reflective configurations, though may be applicable to transmissive configurations.

In some embodiments, the first spatial light modulator 331-a may be used to split the light from the source 310 onto many positions of the object 315. In some embodiments where the first spatial light modulator 331-a modulates the source 310 with a superposition of phase ramps, those phase ramps may Fourier transform to points in the image plane. Thus, the light modulated by a superposition of phase ramps may produce a superposition of light points in the image plane.

In some embodiments, the first spatial light modulator 331-a may be used to adjust the focus of the light from the source 310. A superposition of one or more quadratic phase fronts may be applied to the first spatial light modulator 331-a. These quadratic phase fronts may each generally approximate a lens. Thus, the light from the source 310 may be focused or defocused and, when Fourier transformed, the light at the image plane may be generally focused or defocused. In some embodiments, the first spatial light modulator 331-a modulates with both phase ramps and quadratic phases. In some embodiments, the first spatial light modulator 331-a modulation is reconfigurable. In some embodiments, the first spatial light modulator 331-a modulation is represented by a sequence of controlled, reconfigurable configurations. The reconfigurable configurations may be estimated using a control algorithm, as described below. The control algorithm may be driven by a single signal. The control algorithm may be driven by an image signal.

In some embodiments, the second spatial light modulator 331-b is used to impart a quadratic phase modulation on the wavefront. In some embodiments, the phase front shifts the focus of the beam when imaged onto the specimen 315.

In some embodiments, the second spatial light modulator 331-b modulates a phase across the image that generally compensates for the phase across the specimen surface. As an example, suppose the object 315 has a wedged transmissive media in front of it which may otherwise tilt the focus of the illumination away from the object. In this example, the second spatial light modulator 331-b may provide a phase structure that may compensate for the tilt of the wedge, thereby allowing the illumination to focus on the object 315.

In some embodiments, the presence of the second spatial light modulator 331-b enhances coupling from the specimen 105 onto one or more detection and analysis ports 350. The detection and analysis ports 350 may be PIC input ports. The detection and analysis ports 350 may be fiber coupling input ports for NIR spectroscopy or swept wavelength interferometry. The detection and analysis ports 350 may be an OCT sensor. The detection and analysis ports 350 may be Raman spectroscopy module.

In some embodiments, the components associated with detector port 350 include phase sensitive behavior, as described above. Such devices may include a waveguide, a coherent detector, a coherently detected imaging sensor, and/or the input grating for a photonic integrated circuit, for example. For example, the first spatial light modulator 331-a in the reflective system may also provide a phase modulation that may compensate for the phase modulation of the reflected light such that it may efficiently couple from the many reflected light positions into the phase sensitive device. A single reflected beam may land on the first spatial light modulator 331-a at a position that may image onto the detector port 150. Assuming this reflected beam arrives with a substantial angle; under these conditions, the first spatial light modulator 331-a may apply a linear phase shift such that the light reflected from the first spatial light modulator 331-a is generally angle free; this may enhance the coupling efficiency when the light arrives at the detector port 350.

Various embodiments provide imaging methods for active illumination. For example, a configuration may be utilized in which a desired image may be used to set the phase modulation of the first spatial light modulator 331-a. The desired image may be captured by illuminating the specimen 315 with NIR light, for example, capturing an image of the specimen 315 with a NIR sensitive camera. In some embodiments, the NIR light has light generally between 760 and 800 nm. In some embodiments, the NIR sensitive camera has a bandpass filter to suppress light not within the NIR band. In some embodiments, the NIR sensitive camera has an RGB-IR image sensor. In some embodiments, the position of the NIR image is registered to the adaptive optic system. Registration may include the positional offset and angular orientation of the image with respect to the optical axis of the adaptive optics. Several classes of registration quality may be offered; the registration may be accurate to within a few pixels, the registration may be accurate to a single pixel, and the registration may be to below a pixel, for example.

In some embodiments, the desired image is a stored image. The stored image may be captured from the specimen and a registration sensor may detect the position of the stored image with respect to the adaptive optics. Some embodiments provide the identity of the specimen based on user input, facial recognition, iris recognition, credit card number, and/or other identification methods. Various embodiments use a live camera, a live photodetector, an electrostatic sensor, and/or other sensors to provide an offset measurement for the registration. In some embodiments, the memory is a local device or on a remote server. In some embodiments, the image contains both amplitude and phase information at each resolvable pixel.

In some embodiments, the optimum adaptive optics configuration is recovered from memory. The stored adaptive optics configuration may be solved based on the specimen and a registration sensor detects the position of the stored image with respect to the adaptive optics. Some embodiments provide the identity of the specimen based on user input, facial recognition, iris recognition, credit card number, and/or other identification methods. Various embodiments use a live camera, a live photodetector, an electrostatic sensor, and/or other sensors to provide an offset measurement for the registration. In some embodiments, the memory is a local device or a remote server.

Various embodiments utilize the Gerchberg Saxton method of phase estimation to determine the modulation of the first spatial light modulator 331-a. Because the Fourier transform of the input field is the output field, one may recursively cycle between the input and output field. The Gerchberg Saxton algorithm generally takes the desired image as the forcing function of the input signal. The desired image may comprise of an intensity image, an amplitude image, a phase image, and/or an amplitude plus phase image. The spatial light modulator side of the image may be constrained by the physical properties of the spatial light modulator and the source beam within the system. In some embodiments, the beam profile of the source beam—including but not limited to the position on the spatial light modulator, the amplitude profile of the beam, the phase profile of the beam—is used to constrain the spatial light modulator modulation. In some embodiments, the spatial light modulator properties are used to constrain the spatial light modulator image—including but not limited to the spatial light modulator extent, the quantization of the spatial light modulator, the position of the spatial light modulator relative to the optical axis, the inter-pixel behavior of the spatial light modulator, and/or the spatially dependent vector-based interaction of the spatial light modulator with the input beam.

In some embodiments, the optical properties of the imaging system are used to constrain either the spatial light modulator image, the desired image, or both. The optical properties may include, but are not limited to; the distortion of the imaging system, other aberrations of the imaging system, the spatial distribution of the polarization across the imaging system, and/or the interaction of the desired image with the spatial distribution of the polarization. The spatial light modulator image may be seeded by a stored image. The image may be chosen to optimize the convergence of the Gerchberg Saxton algorithm. In some embodiments, the image is chosen based on the specimen identity. In some embodiments, the SLM is chosen based on a weighted average of prior specimens.

Some embodiments utilize a linear gradient descent to optimize for the spatial light modulator image. One or more reflected signals may be used as the merit function in the gradient descent method. In some embodiments, the linear gradient descent is used as a refinement of the Gerchberg Saxton method. The linear gradient descent may be used to optimize the position and orientation of the desired image with respect to the optical axis. The optimization of the position and orientation may be equivalently applied to the spatial light modulator image position and orientation with respect to the optical axis. The linear gradient descent may then be used for a global optimization of the position and orientation of the desired image as well as the pixel values of the spatial light modulator. In some embodiments, deep learning is to optimize the spatial light modulator image. In some embodiments, a neural network is utilized to optimize the spatial light modulator image.

Some embodiments utilize a second desired image—that of the returned signal on the detector port—to optimize the second spatial light modulator 331-b. In some embodiments, the desired image is generally equal to the output mode structure of the detector port, shifted to match the orientation with respect to the optical axis, modulated by a linear phase to match the tilt with respect to the optical axis, convolved with the point spread function of the imaging system, and/or defocused to reflect the position within the optical system. The position, tilt, point spread function, and defocus may be measured during fabrication. In some embodiments, one or more of these properties are not explicitly measured and instead the spatial light modulator image may be optimized during fabrication. In some embodiments, one or more of properties are measured in real time using an active monitoring sensor.

In some embodiments, the second spatial light modulator image is optimized by fixing the desired specimen image and the second desired image within the Gerchberg Saxton algorithm. When the second spatial light modulator does not shift appreciably with respect to the detector port and with respect to the optical axis, the second desired image generally does not involve refinement. Should a shift occur, one may optimize across shift and orientation of the second desired image. When the desired image shifts, one may optimize across the shift and orientation of the desired image. Some embodiments are configured to monitor the position and orientation of the specimen, modifying the desired image to compensate. In embodiments using the Gerchberg Saxton algorithm, the algorithm may be allowed to converge as long as the specimen may remain generally stable.

In some embodiments, the second spatial light modulator is further optimized following the Gerchberg Saxton algorithm using a gradient descent method where light at one or more detector ports may be monitored to set the merit function. The gradient descent may be used to maximize the light at the desired detector port while minimizing light at one or more other detector ports.

In some embodiments, the second spatial light modulator 331-b is optimized then the first spatial light modulator 331-a is optimized, and then both spatial light modulators are jointly optimized. Both the first spatial light modulator optimization and the joint optimization may use the final coupling of the light into the desired detector port as the measurement to maximize. Some embodiments additionally seek to minimize the light falling on other detector ports. The optimization of the first spatial light modulator 331-a may include the modulation of the second spatial light modulator 331-b. An implementation following the Gerchberg Saxton algorithm for each spatial light modulator 331-a/331-b may be followed by a joint Gerchberg Saxton algorithm. The joint Gerchberg Saxton algorithm may account for the fact that the second spatial light modulator 331-b modulates both transmitted and received light. When the desired output port is generally co-located with the source, the joint optimization may account for the fact that light from both the transmitted and reflected signal are modulated by the first spatial light modulator 331-a.

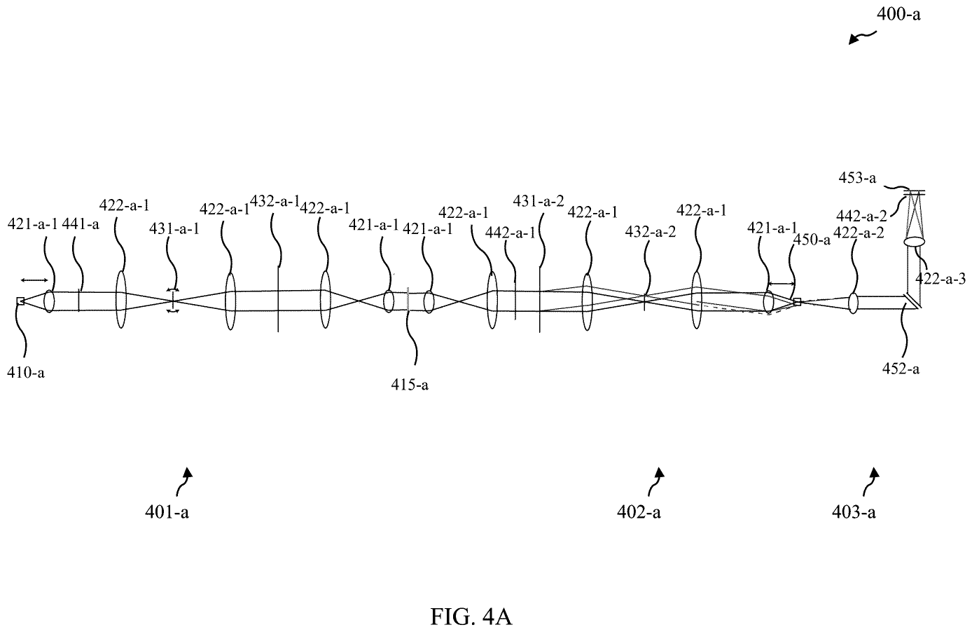

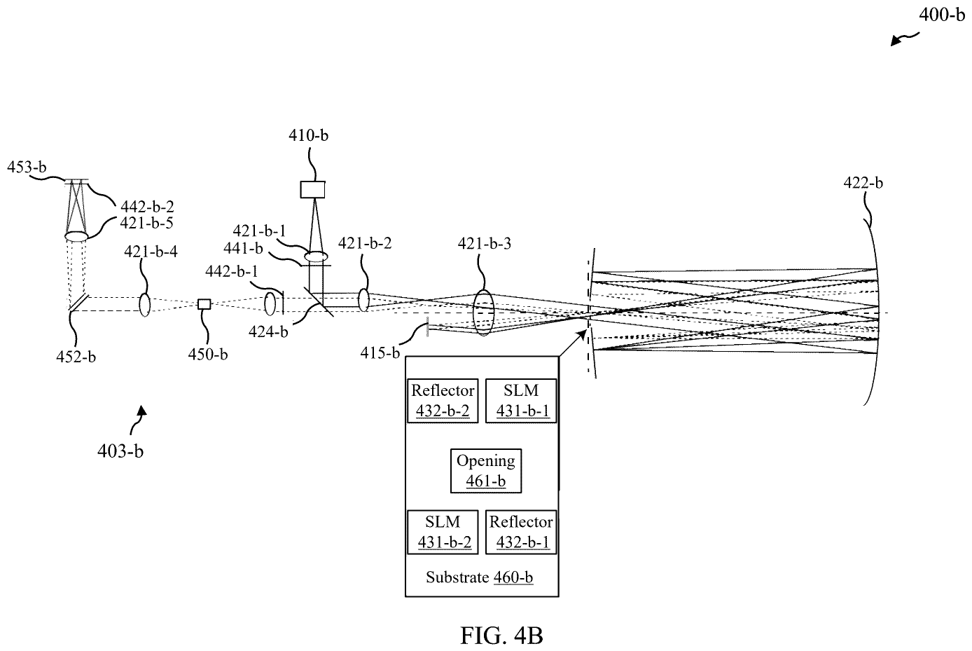

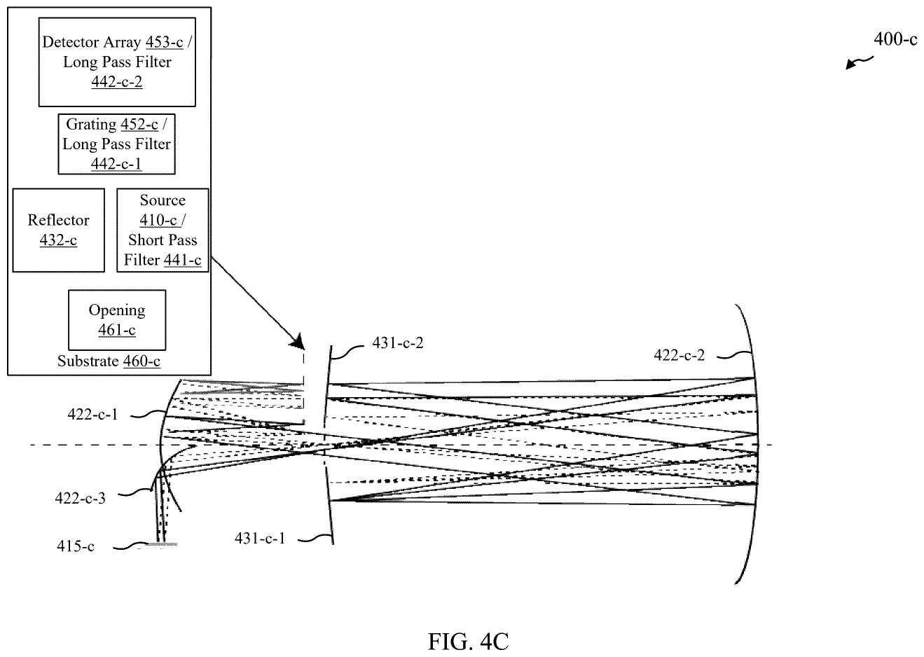

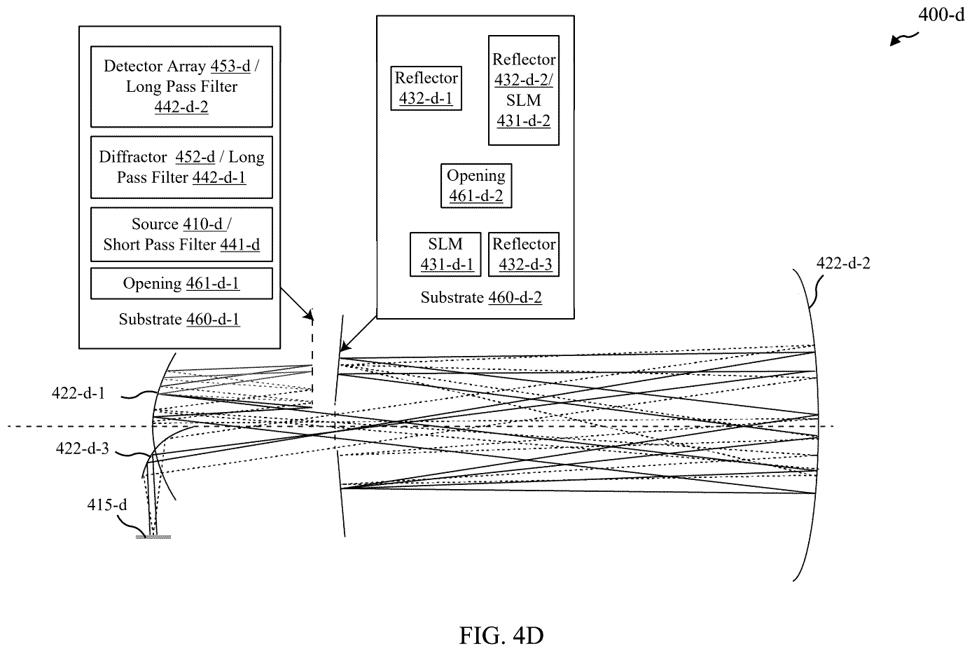

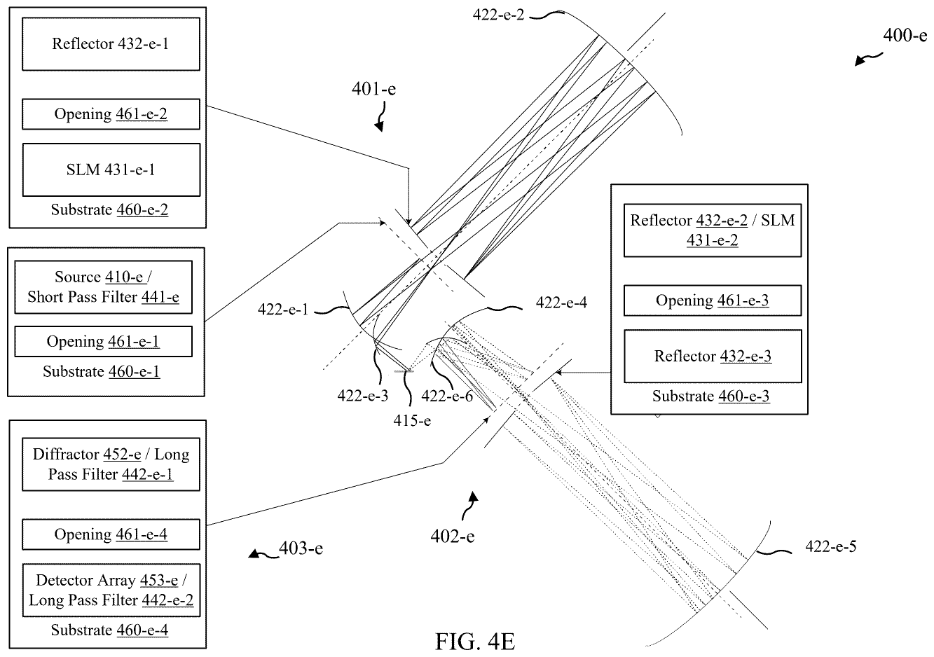

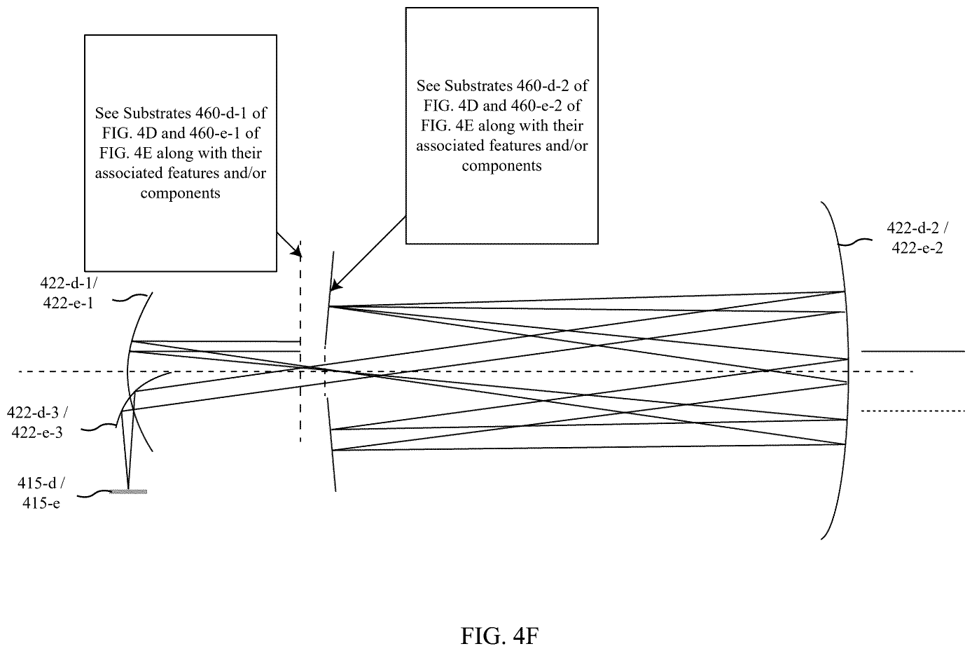

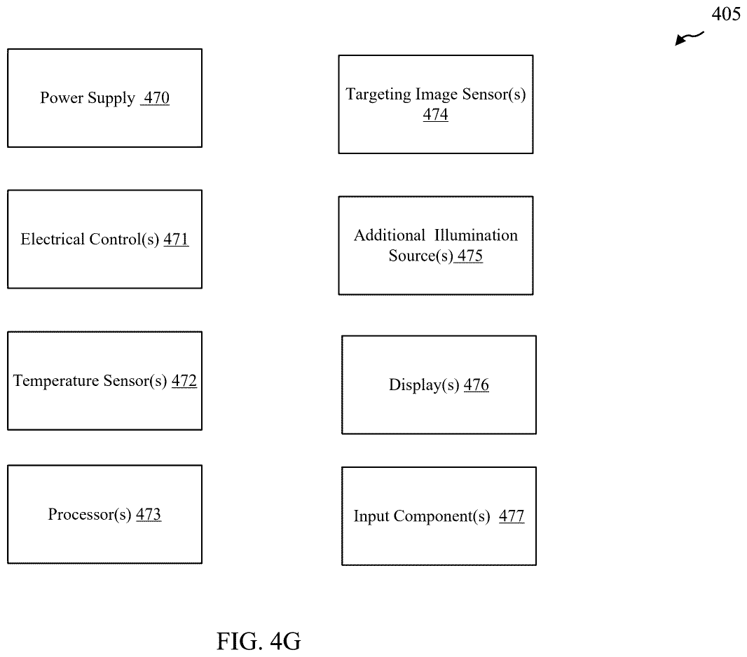

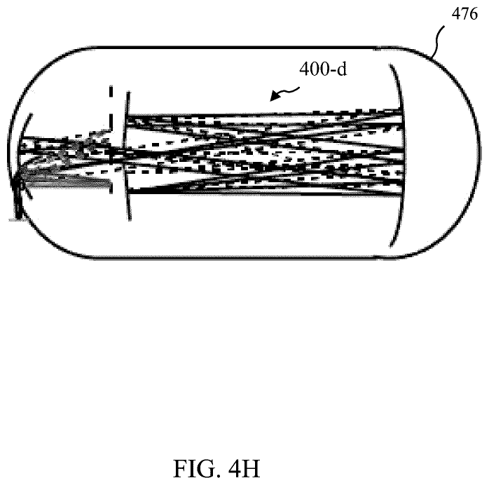

Turning now to FIG. 4A, FIG. 4B, FIG. 4C, FIG. 4D, and FIG. 4E, various systems 400-a, 400-b, 400-c, 400-d, and 400-e are provided in accordance with various embodiments. These systems 400 may be examples of system 100 of FIG. 1. These systems 400 may be configured for the generation of illumination light, the filtering of the illumination light, steering the illumination light, illumination of the object, the collection of the scattered light, filtering of the scattered light from the illuminated light, separating the scattered light into a spectrum, and detecting and digitizing the spectrum.

For example, FIG. 4A shows system 400-a, starting with the light from a source 410-a being generally collimated by a first lens 421-a-1, with a focus that may be adjusted by shifting the source 410-a with respect to the first lens 421-a-1. The collimated light may then be passed through a short pass filter 441-a that generally reduces the longer wavelength light; this may contribute to the system's downstream ability to detect light scattered at these longer wavelengths. The light transmitted through the short pass filter 441-a may then be focused (utilizing concave mirror 422-a-1) onto a MEMS mirror 431-a-1, which is a specific form of spatial light modulator, that may tilt around at least one axis such that the axis may be aligned about the focus of the beam. The light reflected from the MEMS mirror 431-a-1 may then be passed through a Fourier optical system (including concave mirror 422-a-1, reflector 432-a-1 (such as a planar mirror), and lens 421-a) such that an object 415-a may be placed generally at the Fourier plane of the beam focused onto the MEMS mirror 431-a-1. To the object 415-a, these sections of FIG. 4A thus generally show the object 415-a being illuminated by short pass filtered light, and the light may be directed to various positions on the object by tuning the angle of the MEMS mirror 431-a-1. The light may be focused into and out of the object 415-a. In some embodiments, the irradiance of the illumination generally generates a strong Stokes-shifted scattering such that the wavelength of the scattered light is generally shifted to longer wavelengths.

FIG. 4A generally shows the system 400-a configured to collect scattered light from the object 415-a, spectrally filter the light, use adaptive optics to shape the beams, and pass the light into a spectrum analyzer 403-a. From the object 415-a, scattered light may be relayed through a 4-F optical system (which may include lens 421-a-1 and concave mirror 422-a-1) such that the image of the object 415-a may fall onto a reflective spatial light modulator 431-a-2. The spatial light modulator 431-a-2 may be placed behind a long pass filter 442-a-1 such that the light from the illumination beam is generally suppressed relative to the Stokes shifted scatters light. In some embodiments, the spatial light modulator 431-a-2 is programmed with a single blazed grating such that a substantial quantity of the reflected light may be diffracted at an angle. FIG. 4A shows this as two angle-separated reflections from the spatial light modulator 431-a-2. The reflected light may then be passed through a Fourier optical system (which may include reflector 432-a-2 (which may be a planar mirror), concave mirror 422-a-1, and lens 421-a-2) such that the reflected light may be focused generally through the entrance aperture 450-a (or detector port) of the spectrum analyzer. In some embodiments, the spatial light modulator 431-a-2 is programmed with a single blazed grating such that a single mode of scattered light may be passed through the entrance aperture 450-a. In some embodiments, the spatial light modulator 431-a-2 is programmed with a superposition of blazed gratings such that many modes of scattered light may be passed through the entrance aperture 450-a. In some embodiments, the undiffracted light from the spatial light modulator 431-a-2 is passed through the entrance aperture 450-a. The spatial light modulator 431-a-2 may be replaced with a mirror. In some embodiments, the object plane at which light focuses through the entrance aperture 450-a may be shifted by adjusting the position of a second lens. In some embodiments, the spatial light modulator 431-a-2 has a quadratic phase front which shifts the focus. To this point, the scattered light has generally been collected, spectrally filtered, and adaptive optics have been used to select which modes of light pass into the spectrum analyzer 403-a.

With respect to the spectrum analyzer 403-a, the light passing through the entrance aperture 450-a may be passed through a Fourier optical system such that it is generally collimated utilizing concave mirror 422-a-2. The collimated light may then be passed through a fixed grating 452-a such that a substantial portion of the light may be diffracted toward a detector array 453-a. In some embodiments, one may make use of the fact that different wavelengths of light are diffracted at different angles; by placing the diffracted light through a Fourier optical system, the light diffracted at each angle may be focused to a different position. In FIG. 4A, a detector array 453-a may be placed such that each focused beam falls at a different position on the detector array 453-a, utilizing concave mirror 422-a-3. In some embodiments, a long pass optical filter 442-a-2 may be placed in front of the detector array 453-a to further suppress any illumination light which has leaked through the system. The detector array 453-a may be digitized, buffered, and communicated to a processor to analyze the spectra. To this point, one may now have Stokes scattered light focusing onto the detector array 453-a. The position of the light along the detector array 453-a generally corresponds to the wavelength of the light. When multiple wavelengths are present, this embodiment provides a method to measure the scattered light at one wavelength relative to another; more light may produce a stronger detected signal. Thus, the output from the detector array 453-a may be deemed a spectrum analysis of the scattered light. In some embodiments, the spectrum analysis is further enhanced.

Numerous variations on the system 400-a of FIG. 4A may accord with various embodiments. In some embodiments, focus is adjusted by shifting the first lens 421-a. In some embodiments, the short pass filter 441-a is reflective instead of transitive. In some embodiments, the MEMS mirror 431-a-1 is replaced with a spatial light modulator. In some embodiments, the Fourier optical imaging system is constructed of a single lens (a 2-F system), a relay followed by a 2-F system, a 4-F imaging system followed by a 2-F system, or a two lens telecentric 1-F system including a field lens. The lenses of each system may be replaced with concave or convex lenses. In some embodiments, the MEMS mirror 431-a-1 is adjusted by observing the illumination beam through a camera imaging the object 415-a. In some embodiments, the MEMS mirror 431-a-1 is adjusted by observing the spectra produced at the output. In some embodiments, one may collect and analyze the anti-Stokes scattered light.

In some embodiments, the light may not fall at just one spatial position on the detector array 453-a. In some embodiments, one may calibrate the point spread function of the system 400-a and may use this information to deconvolve the image from the detector array 453-a. This deconvolution may enhance the dynamic range of the spectra collected from the detector array 453-a.

In some embodiments, a reflective grating is used. In some embodiments, reflective Fourier optics are used. In some embodiments, transmissive Fresnel optics are used where diffraction and lens functions may be integrated in the same element. In some embodiments, the Fresnel optics are reflective.

FIG. 4A generally shows system 400-a from a propagation path perspective to help facilitate an understanding of the system. System 400-a may be constructed utilizing multiple lenses (similar to system 200-a of FIG. 2A) to create a transmissive system, which may have some advantages in that it may be constructed with widely isolated bulk components. In general, components shown to the left of specimen 415-a in system 400-a may be referred to as illumination components 401-a. In general, components shown to the right of specimen 415-a may be referred to as collection components 402-a until detector port 450-a is reached, which may represent the beginning of the detection components 403-a.

FIG. 4B, FIG. 4C, FIG. 4D, and FIG. 4E generally provide more compact systems by shifting to reflective systems, which may also be referred to as folded systems. FIG. 4B shows how the steering of the illumination beam and the adaptive optics of the collection path may be folded into a reflective system. FIG. 4C, FIG. 4D, and FIG. 4E may show how the source conditioning and the spectrum analysis may be folded into reflective systems.

FIG. 4B shows system 400-b in which the steering of the illumination and the adaptive optics of the scattered light collection may be folded into a reflective system. The scattered light from the object 415-b may be assumed to be collected as a reflection. System 400-b may start with a source 410-b that is generally collimated, utilizing lens 421-b-1, and filtered utilizing a short pass filter 441-b before reflecting off of a dichroic mirror 424-b. This reflection may then be passed through a 4-F system such that the collimated beam (which may be formed utilizing lenses 421-b-2 and 421-b-3) may pass through an opening 461-b in a substrate 460-b. From there, the illumination beam may be passed through a 2-F Fourier system where a convex mirror 422-b may focus the collimated beam into a Fourier plane generally close to the surface of the substrate 460-b. A tunable MEMS mirror 431-b-1 (an example of a spatial light modulator (SLM)) may be placed generally close to the focus of the beam such that it may reflect the focused beam back toward the concave mirror 422-b. The illumination beam reflected from the MEMS mirror 431-b-1 may be collimated by the concave mirror 422-b; it may then reflect from a generally planar mirror 432-b-1 such that the concave mirror 422-b may focus the beam through the opening 461-b in the substrate 460-b. In some embodiments, the focused beam is then Fourier transformed with lens 421-b-3 such that the beam may fall onto the object 415-b. Such a system generally shows how a MEMS mirror 431-b-1 may be used to adjust the angle of the reflection; because this angle occurs in the Fourier plane of the object 415-b, the tilt of the MEMS 431-b-1 may generally translate the position of the beam.

In system 400-b, the scattered light may then be passed back through the Fourier lens 421-b-3 to focus through the opening 461-b in the substrate 460-b. The reflected light may be collimated by the concave mirror 422-b such that the collimated light falls generally on a reflective spatial light modulator 431-b-2. This system 400-b illustrates a spatial light modulator 431-b-2 that may be programmed with a blazed reflective grating such that one beam may be diffracted from the spatial light modulator 431-b-2 to be focused by the convex mirror 422-b onto a generally planar mirror 432-b-2, tilted such that the reflected beam may be generally collimated by the convex mirror 422-b and may pass through the opening 461-b in the substrate 460-b.

The scattered light passing through the substrate 460-b may then be imaged onto the dichroic 424-b that transmits light, which has been Stokes shifted. The dichroic 424-b may remove direct reflections of the illumination beam. The light transmitted through the dichroic 424-b may be passed through a long pass filter 442-b-1 to further suppress the illumination beam. The collimated light may then be focused into the entrance port 450-b of the spectrometer 403-b. The spectrometer 403-b then may collimate (utilizing lens 421-b-4) the beam onto a grating 452-b, the grating 452-b may diffract light from different wavelengths at different angles, and the diffracted light at each angle may then be focused (utilizing lens 421-b-5) onto a specific location on the detector array 453-b. This light may be further cleaned up using an additional long pass filter 442-b-2. This coarse spectrometer may detect, digitize, and communicate the detector array information to a processor for additional processing.

System 400-b generally shows the planar locations of components placed onto the substrate 460-b. At the center may be found an opening 461-b in the substrate 460-b. Opening 461-b may also be referred to as a substrate aperture. MEMS mirror 431-b-1, spatial light modulator 431-b-2, reflector 432-b-1, and reflector 432-b-2 may be mounted on substrate 460-b. Various configurations may be utilized with respect to the placement of these components on substrate 460-b. In some embodiments, the MEMS mirror 431-b-1 and the spatial light modulator 431-b-2 are placed on opposite sides of the opening 461-b, and the reflectors 432-b-1 and 432-b-2 of both the illumination beam and the scattered beam are placed opposite each other. This configuration may collect the largest angular separation between the illumination beam and the scattered light collected. In some embodiments, the MEMS mirror 431-b-1 is placed adjacent to the spatial light modulator 431-b-2. This configuration may collect a different separation of scattering angles from the object 415-b. In some embodiments, multiple spatial light modulators 431-b-2 are placed around a single MEMS mirror 431-b-1; the scattered light may be collected from multiple groups of angles around the illumination beam. Some embodiments, multiple MEMS mirrors 431-b-1 are used to produce multiple illumination beams.

In general, components from source 410-b to specimen 415-b may be considered illumination components, while components from specimen 415-b may be considered collection components. Illumination components and collection may have components in common such as lens 421-b-3, concave mirror 422-b, and/or substrate 460-b. Components from detector port 450-b to detector array 453-b may be considered detection components.

FIG. 4C provides an example of a system 400-c that may form a multi-folded system with respect to illumination and collection. In this example, a first planar substrate 460-c may include a source 410-c covered with a short pass filter 441-c, a mirror 432-c, a grating 452-c covered with a long pass filter 442-c-1, and a detector array 453-c covered with a long pass filter 442-c-2.

In this embodiment, the source 410-c may be collimated by a first convex mirror 422-c-1 before being passed into the opening 461-c of the substrate 460-c and focused utilizing second concave mirror 431-c-2. The steered light from the MEMS mirror or reflective spatial light modulator 431-c-1 and/or reflective spatial light modulator 431-c-2 may be passed through the opening 461-c before being focused onto the object 415-c using a third concave mirror 431-c-3. The scattered light may reflect back across the third concave mirror 422-c-3. The scattered light may be beamformed utilizing reflective spatial modulators 431-c-2 and/or 431-c-1 formed back through the folded system before being passed through the opening 461-c. In some embodiments, the first concave mirror 422-c-1 is at a different plane from the third concave mirror 422-c-3. In some embodiments, the first concave mirror 422-c-1 and the third concave mirror 422-c-3 are co-planar and the first concave mirror 422-c-1 may be truncated. The scattered light returned from the folded system may now be focused by the first concave mirror 422-c-1 onto a thin slit of a reflector 432-c; where the reflector 432-c may serve the same function as the entrance aperture or detector port 450-b to the spectrometer in systems such as system 400-b of FIG. 4B. Light reflected by the slit reflector 432-c may be collimated by the first concave mirror 422-c-1 onto a reflective grating 452-c that may have been covered by a long pass filter 442-c-1 to suppress the illumination beam. Light at different wavelengths may then be diffracted from the reflective grating 452-c at different angles. These different angles may be focused by the first concave mirror 422-c-1 onto different positions along a detector array 453-c. The detector array 453-c may be covered with a long pass filter 442-c-2 to further suppress the illumination beam. This coarse spectrometer may detect, digitize, and communicate the detector array information to a processor for additional processing.

In some embodiments of system 400-c, the focal length of the mirror 422-c-2 is chosen such that the waist at the MEMS mirror 431-c-1 is generally similar to the waist at the spatial light modulator 431-c-2. In some embodiments, the long pass filter 442-c-1 and the grating 452-c may be combined into a single diffractive structure. This structure may be a meta-material; this structure may be a hologram. In some embodiments, the two long pass filters 442-c-1/442-c-2 are the same filter. In some embodiments, the two long pass filters 442-c-1/442-c-2 have different spectral properties. In some embodiments, multiple substates may be utilized, such as utilizing substrate 460-c of FIG. 4C and substrate 460-b of FIG. 4B into system 400-c. In some embodiments, the substrates 460 are fused.

Turning now to FIG. 4D, a system 400-d is shown in accordance with various embodiments. This embodiment provides an example where illumination components shares components with collection components. System 400-d generally includes a light source 410-d, such as a laser. Light from the laser 410-d may diverge from a first focus to form a diverging laser light. A first concave mirror 422-d-1 may be positioned one focal length from the first focus such that the diverging laser light may be reflected from the first concave mirror 422-d-1 as a first collimated light beam. A short pass filter 441-d may be positioned in at least the diverging laser light or the first collimated light beam. One or more substrates 460-d-1/460-d-2 may be positioned one focal length from the first concave mirror 422-d-1; the one or more substrates 460-d may include one or more substrate apertures or openings 461-d-1/461-d-2 such that the first collimated light beam passes through at least one of the one or more substrate apertures 461-d. A second concave mirror 422-d-2 may be positioned two focal lengths from the first concave mirror 422-d-1 such that the second concave mirror 422-d-2 focuses the first collimated light beam to a second focus a first diverging light beam. A MEMs mirror (or more generally a spatial light modulator) 431-d-1 may be positioned at the second focus. The spatial light modulator 431-d-1 may be referred to as a first spatial light modulator. The MEMs mirror 431-d-1 may reflect the first diverging light beam back to the second concave mirror 422-d-2 such that the second concave mirror 422-d-2 collimates the diverging light beam as a second collimated light beam. A first reflector 432-d-1 may be positioned one focal length from the second concave mirror 422-d-2 such that the first reflector 432-d-1 reflects the second collimated light beam off of the second concave mirror 422-d-2 to a third focus within at least one of the one or more substrate apertures 460-d-1/460-d-2 to form a second diverging light beam. A third concave mirror 422-d-3 may be positioned one focal length from the third focus; the third concave mirror 422-d-3 may collimate light from the third focus into a third collimated beam and directs the third collimated beam to an object plane where one or more positions of the object 415-d scatter light back to the third concave mirror 422-d-3 or a fourth concave mirror as one or more diverging object beams such that the one or more first diverging object beams are collimated by the third concave mirror 422-d-3 or the fourth concave mirror to form one or more first collimated object beams that pass through at least one of the one or more substrate apertures 460-d-1/460-d-2 and reflect off the second concave mirror 422-d-2 or a fifth concave mirror to a first object beam focus to form one or more first converging object beams. A second reflector 432-d-2 may be positioned one focal length from the second concave mirror 422-d-2 or the fifth concave mirror, at least a portion of the second reflector 432-d-2 may be positioned at the first object beam focus such that the second reflector 432-d-2 reflects the one or more first converging object beams from the first object beam focus as one or more second diverging object beams to the second concave mirror 422-d-2 or the fifth concave mirror and the second concave mirror 422-d-2 or the fifth concave mirror collimates the one or more second diverging object beams to produce one or more second collimated object beams. In some embodiments, a second spatial light modulator 431-d-2 may be utilized instead of reflector 432-d-2. A third reflector 432-d-3 may be positioned one focal length from the second concave mirror 422-d-2 or the fifth concave mirror; the third reflector 432-d-3 may reflect the one or more second collimated object beams back at the second concave mirror 422-d-2 or the fifth concave mirror such that the one or more second collimated object beams are focused to a second object beam focus within at least one of the one or more substrate apertures 461-d-1/461-d-2 to form one or more third diverging object beams that are directed toward the first concave mirror 422-d-1 or a sixth concave mirror such that the first concave mirror 422-d-1 or the sixth concave mirror collimates the one or more third diverging object beams to form one or more third collimated object beams. A first long pass filter 442-d-1 may be positioned within the one or more third collimated object beams. A diffractor 452-d, such as a diffraction grating, may be positioned one focal length from the first concave mirror 422-d-1 or the sixth concave mirror and intersecting a portion of the one or more third collimated object beams such that the one or more third collimated object beams diffracts according to multiple wavelengths of the one or more third collimated object beams and the multiple wavelengths of the one or more third collimated object beams are directed at the first concave mirror 422-d-1 or the sixth concave mirror such that a diffracted beam at each wavelength from the multiple wavelengths is focused to a different position of the first concave mirror 422-d-1 or the sixth concave mirror to form a spectra of focused beams. A second long pass filter 442-d-2 may be positioned in the spectra of focused beams. A detector array 453-d may be positioned in the spectra of focused beams such that the spectra of focused beams are detected, sampled, and communicated to a processor (see, e.g., FIG. 4G) as a detected spectra.

In some embodiments, the source laser 410-d reflects from the first spatial light modulator 431-d-1 (which may be a MEMS mirror) and is directed to second concave mirror 422-d-2 such that it falls on third concave mirror 422-d-3 without hitting the first reflector 432-d-1. This may allow scanning only in angle. Some embodiments bypass the third concave mirror 422-d-2 for the illumination beam such that steering in position may be enabled. In some embodiments, the third concave mirror 422-d-3 is generally a dichroic and only the scattered light reflects whereas the source may be passed through a second relay.

In some embodiments, the scattered light hits the second reflector 432-d-2 where it is reflected into the substrate aperture 461-d as a collimated beam. When the light hits the first concave mirror 422-d-1, it may be focused. In some embodiments, a fourth reflector on the substrate 460-d is placed at the focus, and may reflect a diverging beam at first reflector 432-d-1 such that it may be collimated and falls onto the diffractor 452-d.

In general, the illumination components and the collection components of system 400-d may share components, such as concave mirrors 422-d-1, 422-d-2, and 422-d-3. Both illumination components and collection components may be found on substrate 460-d-2, while both illumination and detection components may be found on substrate 460-d-1.

Turning now to FIG. 4E, a system 400-e is shown in accordance with various embodiments. This embodiment provides an example where an illumination device 401-e and a collection device 402-e are generally separated from each other. System 400-e generally includes a light source 410-e, such as a laser. Light from the laser 410-e may diverge from a first focus to form a diverging laser light. A first concave mirror 422-e-1 may be positioned one focal length from the first focus such that the diverging laser light may be reflected from the first concave mirror 422-e-1 as a first collimated light beam. A short pass filter 441-e may be positioned in at least the diverging laser light or the first collimated light beam. One or more substrates 460-e-1/460-e-2 may be positioned one focal length from the first concave mirror 422-e-1; the one or more substrates 460-e-1/460-e-2 may include one or more substrate apertures or openings 461-e-1/461-e-2 such that the first collimated light beam passes through at least one of the one or more substrate apertures 461-e-1/461-e-2. A second concave mirror 422-e-2 may be positioned two focal lengths from the first concave mirror 422-e-1 such that the second concave mirror 422-e-2 focuses the first collimated light beam to a second focus a first diverging light beam. A MEMs mirror (or more generally a spatial light modulator) 431-e-1 may be positioned at the second focus. The spatial light modulator 431-e-1 may be referred to as a first spatial light modulator. The MEMs mirror 431-e-1 may reflect the first diverging light beam back to the second concave mirror 422-e-2 such that the second concave mirror 422-e-2 collimates the diverging light beam as a second collimated light beam. A first reflector 432-e-1 may be positioned one focal length from the second concave mirror 422-e-2 such that the first reflector 432-e-1 reflects the second collimated light beam off of the second concave mirror 422-e-2 to a third focus within at least one of the one or more substrate apertures 461-e-1/461-e-2 to form a second diverging light beam. A third concave mirror 422-e-3 may be positioned one focal length from the third focus; the third concave mirror 422-e-3 may collimate light from the third focus into a third collimated beam and directs the third collimated beam to an object plane where one or more positions of the object 415-e scatter light back to a fourth concave mirror 422-e-4 as one or more diverging object beams such that the one or more first diverging object beams are collimated by the fourth concave mirror 422-e-4 to form one or more first collimated object beams that pass through at least one of one or more substrate apertures 461-e-3/461-e-4 of substrates 460-e-3/460-e-4 and reflect off a fifth concave mirror 422-e-5 to a first object beam focus to form one or more first converging object beams. A second reflector 432-e-2 may be positioned one focal length from the fifth concave mirror 422-e-5, at least a portion of the second reflector 432-e-2 may be positioned at the first object beam focus such that the second reflector 432-e-2 reflects the one or more first converging object beams from the first object beam focus as one or more second diverging object beams to the fifth concave mirror 422-e-5 and the fifth concave mirror 422-e-5 collimates the one or more second diverging object beams to produce one or more second collimated object beams. In some embodiments, a second spatial light modulator 431-e-2 may be utilized instead of reflector 432-e-2. A third reflector 432-e-3 may be positioned one focal length from the fifth concave mirror 422-e-5; the third reflector 432-e-3 may reflect the one or more second collimated object beams back at the fifth concave mirror 422-e-5 such that the one or more second collimated object beams are focused to a second object beam focus within at least one of the one or more substrate apertures 461-e-3/461-e-4 to form one or more third diverging object beams that are directed toward a sixth concave mirror 422-e-6 such that the sixth concave mirror 422-e-6 collimates the one or more third diverging object beams to form one or more third collimated object beams. A first long pass filter 442-e-1 may be positioned within the one or more third collimated object beams. A diffractor 452-e, such as a diffraction grating, may be positioned one focal length from the sixth concave mirror 422-e-6 and intersecting a portion of the one or more third collimated object beams such that the one or more third collimated object beams diffracts according to multiple wavelengths of the one or more third collimated object beams and the multiple wavelengths of the one or more third collimated object beams are directed at the sixth concave mirror 422-e-6 such that a diffracted beam at each wavelength from the multiple wavelengths is focused to a different position of the sixth concave mirror 422-e-6 to form a spectra of focused beams. A second long pass filter 442-e-2 may be positioned in the spectra of focused beams. A detector array 453-e may be positioned in the spectra of focused beams such that the spectra of focused beams are detected, sampled, and communicated to a processor (see, e.g., FIG. 4G) as a detected spectra.

In general, the illumination components of system 400-e generally include substrate 460-e-1 with its various features and components, substrate 460-e-2 with its various features and components, and concave mirrors 422-e-1, 422-e-2, and 422-e-3. Collection components generally include substrate 460-e-3 and its various features and components and concave mirrors 422-e-4, 422-e-5, and 422-e-6. Detection components 403-e generally include substrate 460-e-4 and its various features and components.

The illumination beam at the object 415-d/415-e typically is delivered with sufficient irradiance such that the Raman signal may be detected and digitized by the spectrometer, such as diffractor 452-d/452-e and detector array 453-d/453-e. FIG. 4D and FIG. 4E generally show how a diverging laser source 410-d/410-e may be steered onto the object 415-d/415-e as a collimated beam. Such a condition may be delivered if the focal lengths of the first concave mirror 422-d-1/422-e-1, the second concave mirror 422-e-2/422-e-2, and third concave mirrors 422-d/3/422-e-3 are jointly constrained. Such a condition may also involve tradeoffs between the substrate aperture(s) 461 size(s) and the size of first spatial light modulator 431-d-1/431-e-2. These conditions may limit the design.

FIG. 4F provides an example of how illumination components of systems 400-d and 400-e may be utilized to address these issues. In FIG. 4F, the laser source 410-d/410-e may form a collimated laser source in the systems 400-d and/or 400-e; similar modifications may be made with respect to systems 200 of FIGS. 2, systems 300 of FIG. 3, and/or systems 400 of FIGS. 4A, FIG. 4B, and/or FIG. 4C. The collimated laser 410-d/410-e may be directed at first concave mirror 422-d-1/422-e-1 such that the reflected beam converges to a focus within the substrate apertures 461-d/461-e (see substrates 460-d-1, 460-d-2, 460-e-1, 460-e-2 and their associated features and/or components from FIG. 4D or FIG. 4E). The beam may diverge from the focus within the substrate apertures 461-d/461-e to reflect off of second concave mirror 422-d-2/422-e-2 such that the reflected beam is collimated and directed at an angle toward reflective spatial light modulator 431-d-1/431-e-1, which may reflect the collimated beam at a steerable angle directed at the second concave mirror 422-d-2/422-e-2. As the collimated and directed beam hits second concave mirror 422-d-2/422-e-2, it may be reflected to focus at a position on a first reflector 432-d-1/432-e-1, which reflects the focused beam as a diverging beam directed at the second concave mirror 422-d-2/422-e-2. The diverging beam may reflect from the second concave mirror 422-d-2/422-e-2 such that the beam is collimated and directed through the substrate apertures 461-d/461-d to land on third concave mirror 422-d-3/422-e-3. The collimated beam may reflect from third concave mirror 422-d-3/422-e-3 such that it is focused at the specimen 415-d/415-e.

To reiterate, the laser 410-d/410-e may be configured such that light from the laser 410-d/410-e is collimated at a first focus to form a collimated laser light. The first concave mirror 422-d-1/422-e-1 may be positioned one focal length from the first focus such that the collimated laser light is focused by the first concave mirror 422-d-1/422-e-1 to a second focus as a first converging light beam. The short pass filter 441-d/441-e may be positioned in at least the collimated laser light or the first converging light beam. The one or more substrates 460-d-1/460-e-1 may be positioned one focal length from the first concave mirror 422-d-1/422-e-1; the one or more substrates 460-d-1/460-e-1 may include one or more substrate apertures 461-d/461-e such that the first converging light beam passes through at least one of the one or more substrate apertures 461-d/461-e The second concave mirror 422-d-2/422-e-2 may be positioned two focal lengths from the first concave mirror 422-d-1/422-e-1 such that the second concave mirror 422-d-2/422-e-1 collimates the first converging light beam to a third focus as a second collimated light beam. The MEMs mirror 431-d-1/431-d-2 may be positioned at the third focus; the MEMs mirror 431-d-1/431-e-2 may reflect the second collimated light beam back to the second concave mirror 422-d-2/422-e-2 such that the second concave mirror 422-d-2/422-e-2 focuses the second collimated light beam as a second converging light beam. The first reflector 432-d-1/432-e-2 may be positioned one focal length from the second concave mirror 422-d-2/422-e-2 such that the first reflector 432-d-1/432-e-1 reflects the second converging light beam off of the second concave mirror 422-d-2/422-e-2 to a fourth focus within at least one of the one or more substrate apertures 461-d/461-e to form a third collimated light beam. The third concave mirror 422-d-3/422-e-3 may be positioned one focal length from the fourth focus, where the third concave mirror 422-d-3/422-e-3 focuses light from the third collimated light beam into a third converging beam and directs the third converging beam to an object plane.

In some embodiments, the steerable angle of the first spatial light modulator 431-d-1/431-e-1 corresponds to the position on first reflector 432-d-1/432-e-2 and the position on the specimen 415-d/415-e. The steerable angle may be controlled by programing one or more gratings onto the first spatial light modulator 431-d-1/431-e-1. In some embodiments, the first spatial light modulator 431-d-1/431-e-1 is a MEMS mirror, and the steerable angle may be controlled by controlling the angle of the MEMS mirror.

The irradiance of a focused beam at the specimen 415-d/415-e may be sufficient to deliver a detectable Raman spectra. The illumination and beam steering provided through FIG. 4F may replace the methods shown elsewhere herein.

The illumination and beam steering in FIG. 4F may be enabled by replacing the diverging laser 410-d/410-e in FIG. 4D or FIG. 4E with a collimated laser. This replacement of the diverging laser may also involve changes to the focal lengths of one or more concave mirrors 422-d/422-e. This replacement may involve design changes to the sizes of first spatial light modulator 431-d-1/431-e-1 and the substrate apertures 461-d/461-e.

In some embodiments, the laser 410-d/410-e and the short pass filter 441-d/441-e are mounted on the same substrates, such as substrates 460-d-1 and 460-e-1. In some embodiments, the first long pass filter 442-d-1, the diffractor 452-d, the second long pass filter 442-d-1, and the detector array 453-d are mounted the substrate 460-d-1. In some embodiments, the first long pass filter 442-e-1, the diffractor 452-e, the second long pass filter 442-e-1, and the detector array 453-e are mounted another substrate 460-e-4. In some embodiments, the first spatial light modulator 431-d-1/431-e-1 and the first reflector 432-d-1/432-e-1 are mounted to the same substrates 460-d-2/460-e-2. In some embodiments, the second reflector 432-d-2 (or second spatial light modulator 431-d-2) and the third reflector 432-d-3 are mounted on substrate 460-d-2. In some embodiments, the second reflector 432-e-2 (or second spatial light modulator 431-e-2) and the third reflector 432-e-3 are mounted on substrate 460-e-3.

In some embodiments, the laser 410-d/410-e includes an array of lasers and the MEMS mirror 431-d-1/431-e-1 is a spatial light modulator. The array of lasers may include a VCSEL array.

In some embodiments, a volume separating at least the first concave mirror 422-d-1/422-e-1, the second concave mirror 422-d-2/422-e-2, or the third concave mirror 422-d-3/422-d-3 from a plane of the at least one of the substrates 460-d-1/460-d-2 or 460-e-1/460-e2 is filled with a material transparent at a wavelength of the laser 410-d/410-e. The material filling the volume may be safe for use in-vivo with biological systems.

In some embodiments, the wavelength of the laser 410-d/410-e is stabilized to less than 2 nm. The short pass filter 441-d/441-e may include a passband aligned to the wavelength of the laser 410-d/410-e that rejects light more than 20 nm longer than the wavelength of the stabilized laser 410 by at least 30 dB; the first long pass filter 442-d-1/442-e-1 may reject light at the wavelength of the laser 410-d/410-e by at least 30 dB and may pass light more than 20 nm from the wavelength of the laser 410-d/410-e. The second long pass filter 442-d-2/442-e-2 may reject light at the wavelength of the laser 410-d/410-e by 30 dB and may pass light more than 20 nm from the wavelength of the laser 410-d/410-e.

Some embodiments include a biologically inert and optically transparent shell 476 that houses the system 400 such that the system 400 is configured to be implanted into a person. See FIG. 4H, for example, which may show transparent shell 476 around system 400-e. The system 400 may be configured to be placed under the skin of a person. Other systems 400 of FIG. 4 may be similarly configured.

Systems 400 may include additional components. For example, FIG. 4G includes a subsystem 405 of components that may be included in systems 400 of FIG. 4A-FIG. 4F. This may include one or more power supplies 470, one or more electrical controls 471, one or more temperature sensors 472, and/or one or more processors 473. One or more of these components may be configured such that: a current driving the laser 410 is configured to be controlled; a temperature of the laser is configured to be controlled; and/the MEMS mirror 431 is configured to be tilted along one or more axes.

Some embodiments of subsystem 405 may include one or more targeting image sensors 474 and/or one or more additional illumination sources 475. In some embodiments of systems 400, the first reflector is a long pass filter positioned in front of the targeting image sensor 474 such that the long pass filter reflects light from the laser while transmitting light at longer wavelengths than the laser to the targeting image sensor 474. The targeting image sensor 474 may be positioned in an image plane of the object and collects one or more images using light from the second illumination source 475.

In some embodiments of subsystem 401, the targeting image sensor 474 is configured to be powered on; the targeting image sensor 474 is configured to be configurable; one or more settings of the targeting image sensor 474 are configured to be communicated to at least the processor 473 or the electrical controls 471; the image from the targeting image sensor 474 is configured to be communicated to the processor 473; the spatial light modulator is configured to be powered on; the spatial light modulator is configured to be configurable; one or more settings of the spatial light modulator are configured to be communicated to at least the processor 473 or the electrical controls 471; and a value of one or more temperature sensors 472 is configured to be communicated to at least the processor 473 or the electrical controls 471.

In some embodiments, one or more displays 476 are provided to display various information to a user, such as measurement options and/or results. One or more input components 477 may also be provided to take further input from a user.

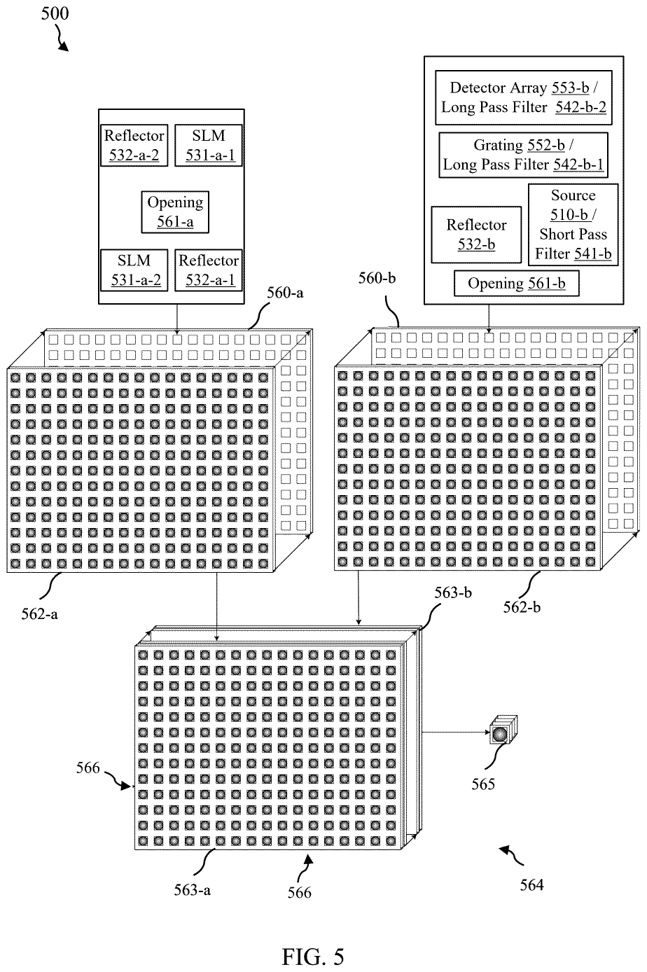

In some embodiments, systems 400 are manufactured where one step of the manufacturing is the dicing of a multi-layered wafer comprising: a spectrometer integrated optic layer; a spectrometer substrate layer; a steering substrate layer; and a steering integrated optic layer. See FIG. 5, for example.

Turning now to FIG. 5, an example of system 500 with one or more substrates 560 are provided in accordance with various embodiments. System 500 may be an example of system 100 of FIG. 1. Some particular features of these various embodiments include the ability to integrate the several electro-optic components onto the one or more substrates. These methods may enable the construction of various devices using either wafer-level optics fabrication and/or integrated electronics fabrication techniques. FIG. 5 shows examples of how large numbers of Raman spectroscopy devices may be manufactured in parallel.

For example, one substrate 560-a may be constructed with hundreds of MEMS mirrors/spatial light modulators 531-a-1 reflectors 532-a-1/532-a-2, openings 561-a and/or spatial light modulators 531-a-2. Substrate 560-a may be referred to as a steering substrate. In some embodiments, the steering substrate 560-a s bonded to a steering integrated optic 562-a that may be reflective and may deliver one first concave mirror to each grouping of MEMS mirrors, mirrors, openings, and spatial light modulators. The alignment of the steering integrated optic 562-a to the steering substrate 560-a may include an active alignment driven by an optical sensor. The alignment may be aided by alignment fiducials placed on both the steering substrate 560-a and the steering integrated optic 562-a. The integration of the steering substrate 560-a to the steering integrated optic 562-a delivers a steering wafer 563-a. When the steering substrate 560-a may be connected electrically, each steering system in the steering wafer 563-a may be tested and validated without dicing.

Some embodiments include a second substrate 560-b that may be constructed with hundreds of sources 510-b, gratings 552-b, long pass filters 452-b-1/452-b-2, short pass filters 541-b, reflectors 532-b and/or detector arrays 553-b. This may be referred to as the spectrometer substrate. The spectrometer substrate 560-b may be bonded to a spectrometer integrated optic 562-b that may be reflective and deliver one second concave mirror and one object mirror to each grouping of sources, gratings, long pass filters, short pass filters, and/or detector arrays. The alignment of the spectrometer integrated optic 562-b to the spectrometer substrate 560-b may include an active alignment driven by an optical sensor. The alignment may be aided by alignment fiducials placed on both the spectrometer substrate 560-b and the spectrometer integrated optic 562-b. The integration of the spectrometer substrate 560-b to the spectrometer integrated optic 562-b may deliver a spectrometer wafer 563-b. When each substrate 560-b can be connected electrically, each spectrometer in the spectrometer wafer 563-c may be tested and validated without dicing.

Some embodiments include the integration of the steering wafer 563-a with the spectrometer wafer 563-b as may be shown in the bottom half of FIG. 5. This may be done by bonding the back side of the steering substrate 560-a to the back side of the spectrometer substrate 560-b. In some embodiments, the two substrates 560-a/560-b are bonded to a third substrate. The alignment of the two wafers 563-a/563-b may be done using fiducials placed on the spectrometer substrate 560-b and the steering substrate 560-a. The fiducials may be used alongside an optical alignment technique. The aligned and bonded stack of a spectrometer wafer 563-b and a steering wafer 563-b may be called a Raman device wafer 564. A resulting single device 565 may also be shown along with dice 566 between reflectors on wafer 563-a

Many Raman devices 565 may be constructed from a Raman device wafer 564. This may be done by dicing the Raman devices. For example, one or more surfaces of the Raman device may be further polished in a secondary step. The electrical connections may be added to the Raman devices. In some embodiments, the Raman devices are connected to a power circuit including a power source. The Raman devices may be encapsulated in a biologically compatible shell. In some embodiments, the Raman devices may be inserted beneath the skin.

FIG. 6 provides an example of an integration of adaptive optic structure 600 that may be implemented using an integrated optical network in accordance with various embodiments. Structure 600 may be an example of system 100 of FIG. 1. The top portion of FIG. 6 generally shows photonics chip 600 may be integrated between many sensing chips to couple light into 688 and out of a patient 680. The photonic chip 600 may include controller chips 680, CP chip 681, and one or more analysis chips like blood 02 chip 682, cholesterol chip 683, glucose chip 684, and/or progesterone/testosterone chip 685. Photonic chip 600 may also include an organic substrate 686 that may include a sensor window 687. The bottom of FIG. 6 generally shows a way of constructing an adaptive network for transmit (or receive) contains a fan-in/fan-out set of splitters 690, a series of delay lines 691, a Benes mixing network 692, and an array of couplers 693.

For example, a set of analysis chips may be arranged to couple into the adaptive optics chip. The analysis section may include a single port input and a single port output; some embodiments include one or more of the input or output ports that may be built with many ports. The transmitted beam may be split into an array of delay lines. Each delay line may be fed into one port of a mixing network. The mixing network may be illustrated as a series of 2×2 couplers in which the input may be split into two outputs and in which the splitting ratio may be controlled. Through a cascaded series of 2×2 couplers, one may achieve any arbitrary mixing of the delay lines desired for a set of output couplers. The output couplers then may emit the light which passes through a window into the specimen.

FIG. 6 generally shows light from the specimen radiating back toward the collection network. At the collection network, reflected or transmitted light may enter the collection network through a set of output couplers. Each of the output couplers may then be passed through a mixing network such that the output may contain an arbitrarily weighted and summed set of the fields at each and every output coupler. Each output from the mixing network may then be passed through the delay line network. The outputs from the delay line may be passed into a series of combiners such that one or more detector ports may be available to be fed into an analysis device. When properly optimized, the superposition of fields output from the mixing network may generally increase the output light levels provided to the analysis device.

In some embodiments, these analysis chips contain a source or the source may be placed elsewhere in the system. The analysis chips may be manufactured on the same substrate. In some embodiments, the analysis chips are packaged into a single substrate and coupled to the adaptive optics chip. In some embodiments, the coupling between chips are performed by butt-coupling the waveguides for the two chips together. The waveguides may be expanded on one or more of the chips to loosen the tolerances of assembly. In some embodiments, the alignment of the chips is done through active alignment.

In some embodiments, the delay of each delay line is a linear function of the index. In some embodiments, the delay line increases logarithmically with the index. In some embodiments, the delay line increases exponentially.

In some embodiments, the 2×2 couplers is controlled by electro-optic modulation of a waveguide. In some embodiments, small heaters drive a thermo-optic effect to control the 2×2 couplers.

In some embodiments, the mixing network is a Benes configuration. In some embodiments, the mixing network is a sparse representation of the Benes configuration.

In some embodiments, the output couplers are Bragg beamsplitters. In some embodiments, the output couplers are coupled out of plane through an angled cut faced that may induce total internal reflection on the beam. In some embodiments, the output couplers are waveguides drawn to the edge of a photonic integrated circuit.

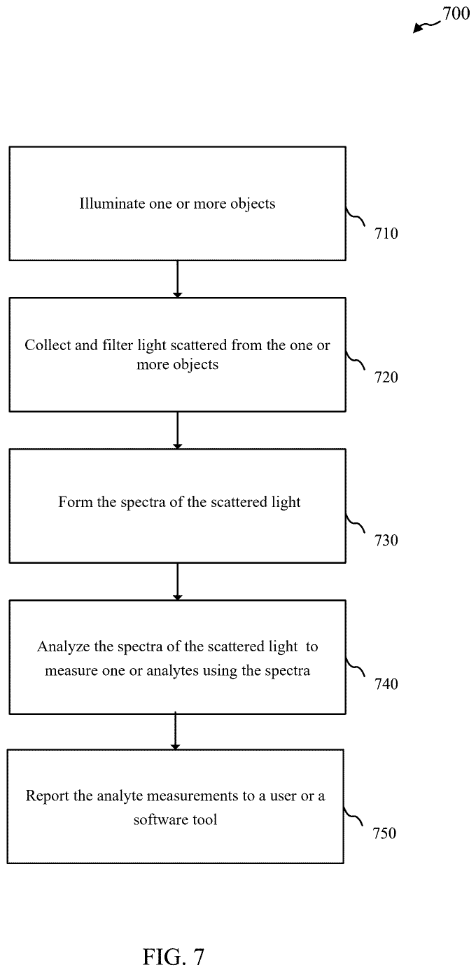

FIG. 7 provides a flow diagram of a method 700 in accordance with various embodiments. Method 700 may be implemented utilizing a variety of systems and/or devices shown and/or described with respect to FIG. 1, FIGS. 2, FIG. 3, FIGS. 4, FIG. 5, and/or FIG. 6.

At block 710, one or more objects may be illuminated. At block 720, light scattered from the one or more objects may be collected and filtered. At block 730, the spectra of the scattered light may be formed. At block 740, the spectra of the scattered light may be analyzed to measure one or analytes using the spectra. At block 750, the analyte measurements may be reported to a user or a software tool.

Method 700 may include delivering analyte measurements of the biological processes within a person or living system where the analyte measurements may provide a useful indicator of the health or wellness of the person or living system. Method 700 may deliver higher sensitivity and a larger tolerance to stray light than may be produced using other methods.

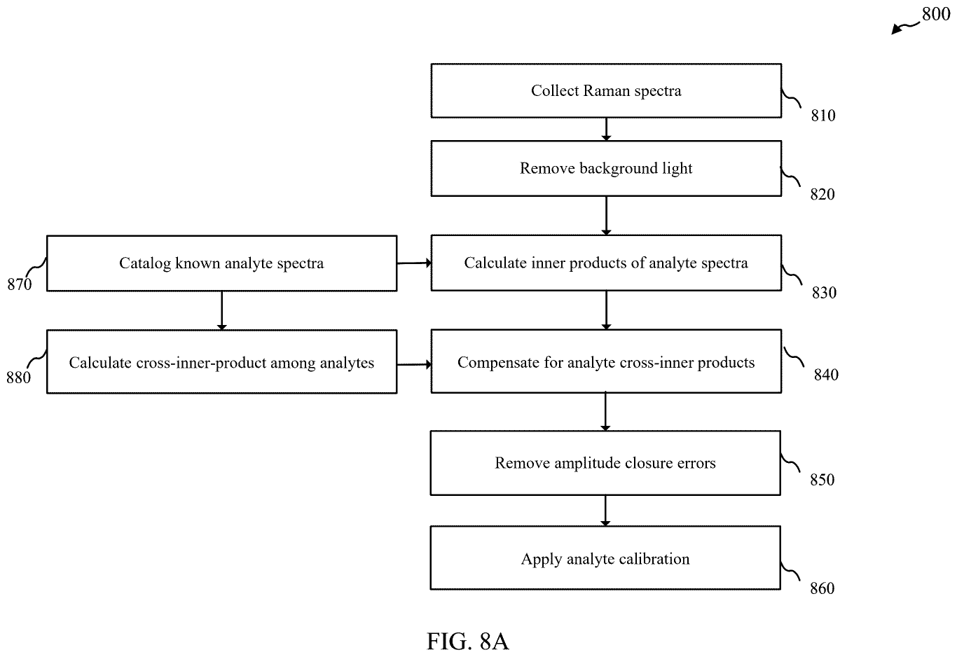

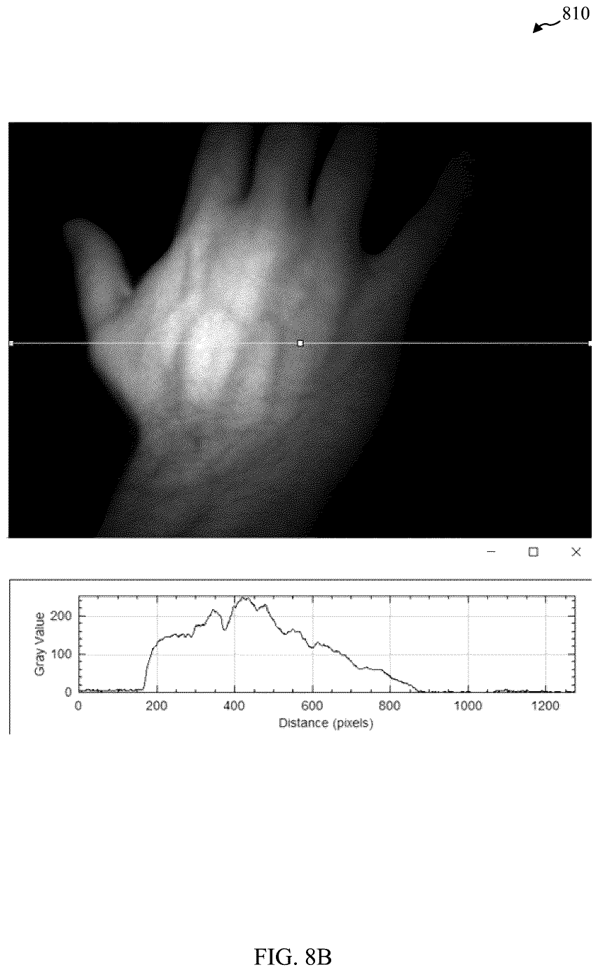

FIG. 8 provides a flow diagram of a method 800 in accordance with various embodiments. Method 800 may be implemented utilizing a variety of systems and/or devices shown and/or described with respect to FIG. 1, FIGS. 2, FIG. 3, FIGS. 4, FIG. 5, and/or FIG. 6. Method 800 may be an example of aspects of method 700 of FIG. 7. Method 800 may include a method to measure analytes from the spectra produced by a spectrometer.

At block 810, a Raman spectra may be collected. The Raman spectra may be detected, digitized, processed, and/or stored within memory. The Raman spectra may be a collection of measured levels and the spectral value at which the measured values occur. The Raman spectra may be calibrated and resampled such that the increment between each spectral value may be uniform. The spectral value may be recorded as the wavenumbers relative to the calibrated wavelength, and the calibrated wavelength may generally equal to the illumination wavelength of the light source, such as a laser. In some embodiments, the Raman spectra is uncalibrated, and the calibration may generally be applied before proceeding with the following processing steps.

At block 820, background light may be removed. For example, once the calibrated Raman spectra has been collected, steps may be taken to remove the background signal from the Raman spectra. The background may be removed by performing a polynomial fit to the spectra. Other embodiments use other fits such as the “bubble fit”. The background may be removed by measuring a reference Raman spectra at a different location on the object and subtracting this reference Raman spectra from the Raman spectra. The removal of the background from the Raman spectra may produce a bias-free spectra.

At block 870, known analyte spectra may be cataloged. For example, some embodiments analyze the Raman spectra using a priori knowledge of the substances within the sample. A catalog of the bias-free spectra for many substances within the sample may be utilized. Each spectra may be adjusted such that the calibration of the hardware and the resolution of the hardware deliver a catalog of bias-free spectra that may generally be identical to how the substances appear within an object placed within the system. In some embodiments, the catalog of bias-free spectra are adjusted each time they are pulled from the catalog. In some embodiments, the catalog of bias-free spectra are not adjusted. In some embodiments, only a subset of the spectra within the catalog are pulled. In some embodiments, the selection of this subset of spectra is varied, and the selection may be evaluated as a part of an optimization process.

At block 830, the inner products of the analyte spectra may be calculated. At block 880, cross-inner products among analytes may be calculated. At block 840, analyte cross-inner products may be compensated for. For example, the catalog of spectra may be further processed to calculate the inner product of each spectra with respect to all other spectra. This collection of cross inner products may be used to compensate for the cross inner products that may occur when processing the bias-free spectra from each sample. The inner product between the bias-free spectra for the sample with each of the substances in the catalog may be calculated. This may produce a vector of inner products. The vector of inner products may then be processed to remove the impact of the cross inner-products for each known analyte. In some embodiments, the compensation is a simple, deterministic matrix multiplication as discussed further below. In some embodiments, the compensation is an iterative, model based method that may converge on the revised weights. The output from the process may be the multi-analyte compensated inner product vector—or MA vector, for short. Steps may be taken to compensate for variations in the amplitudes in one MA vector with respect to other MA vectors collected from the same source. In some embodiments, two or more MA vectors are compared using an amplitude closure relationship described further below.

At block 850, amplitude closure errors may be removed. For example, amplitude closure relationship generally allow for a processor to solve for errors in the amplitudes of two or more MA vectors. The errors can then be removed from the MA vector to produce two or more self-calibrated vectors of measurements—where each may be referred to as an SC vector. In some embodiments, the SC vector is constructed by applying the amplitude closure relationship to two or more vectors of inner products.

At block 860, analyte calibration may be applied. For example, the MA vector may need to be converted into a set of analyte measurements. As the values at this point may have been constructed based solely on the Raman spectra, they may need to be mapped into units with analyte meaning—such as ug/dL (micro-grams per deciliter). This may be done by applying the analyte calibration to each measurement. In some embodiments, each analyte has a linear calibration curve consisting of an offset and a scale factor, and the calibration curve is unique to each analyte. In some embodiments, each analyte has a nonlinear calibration curve.

The calibration curve for one individual may be identical to the calibration curves of multiple individuals. In some embodiments, the calibration curve for one individual varies from that of other individuals. In some embodiments, the calibration curve is measured directly for each individual. In some embodiments, a single reference calibration curve is adjusted to match the data of an individual using amplitude closure relationships between individuals. The resulting output from method 800 may include a vector or analyte measurement.

Some methods in accordance with various embodiments deviate from those described by method 800. In some embodiments, the inner product calculation is replaced by a fitting routine in which the minimum mean square error between a model and the measurement may be found. In some embodiments, the model is simply a weighted summation of analyte spectra from the catalog. One may choose to forego the method to remove the impact of the cross inner-products for each known analyte. In some embodiments, the inner product is further normalized by the total energy within the spectra. In some embodiments, the mean of the background is used to normalize the bias-free spectra; this method may attempt to compensate for environmental variations in amplitude. In some embodiments, one or more amplitude levels from a targeting image sensor are used to normalize the bias-free spectra.

Method 800 and other methods in accordance with various embodiments may include analyte-based self-calibration. Self-calibration methods may look for properties within the data—such as differential measurements or measurement ratios—that are consistent across datasets. The methods then may use these consistency checks to further constrain models of the measurement data. The use of amplitude ratios for a self-calibration algorithm are discussed below.

For example, the signal spectra may be modelled as a weighted sum of the analyte spectra a\vec{ }. The quantity of each analyte may be measured by estimating the collection of all composition weights cᵢ∀i∈N.

$${S}^{\to } = {\sum }_{i = 1}^{N} {c}_{i}^{′} {a}_{i}^{\to }$$

With spectra from multiple locations or from multiple individuals, one may build up a collection of these signals, each with an index according to the location 1 and the subject k. The weight of each analyte

$${c}_{i}^{′} = {c}_{l} {c}_{k} {c}_{i}$$

may be decomposed into weights associated with (i) the analyte, (l) the location, and (k) the individual.

$${S}_{lk}^{\to } = {c}_{l} {c}_{k} {\sum }_{i = 1}^{N} {c}_{i} {a}_{i}^{\to }$$

The inner product of the signal may be calculated with the spectra of any one analyte:

$${P}_{S} ( j , k , l ) = {S}_{lk}^{\to } \cdot {a}_{j}^{\to } = {c}_{l} {c}_{k} {\sum }_{i = 1}^{N} {c}_{i} {d}_{ij}$$

where the inner product of the signal may now be expressed as a weighted sum of inner products dᵢⱼ=aᵢ\vec{ }·aⱼ\vec{ } for one analyte with all other analytes. The collection of all known analyte inner products may be expressed as a reference inner product matrix D with cells indexed by rows j and columns i are filled with dᵢⱼ. The composition weights may be rolled into a vector representation C where each element is cᵢ. When the library matrix L (where each row is the spectra of one analyte) is multiplied by the signal vector S, the result may be the inner product matrix multiplied by the composition weights.

$${\underline{P}}_{lk} = \underline{\underline{L}} {\underline{S}}_{lk} = \underline{\underline{D}} {\underline{C}}_{lk}$$

This equation may be utilized to solve for the composition weights as:

$${\underline{C}}_{lk} = {\underline{\underline{D}}}^{- 1} {\underline{P}}_{lk}$$

Such a solution may deliver the solution for all of the composition weights, and D, the collection of inner products among all pairs of analytes, may be calculated. This result may provide results across many analytes, many locations, and/or many individuals.

Some embodiments provide for self-calibration between sites on a single individual. For example, the methods of self-calibration may be adapted to the various datasets. In self-calibration, the ratio of the four relative amplitudes between four different points may be a constant. One may take advantage of a similar property. For any two analytes compared across any two sites, four separate composition weights may be provided. The ratio of these four composition weights may be:

$${R}_{abyzkk} = \frac{{C}_{ayk} {C}_{bzk}}{{C}_{azk} {C}_{byk}} = \frac{{\underline{D}}_{a}^{- 1} {\underline{P}}_{yk} {\underline{D}}_{b}^{- 1} {\underline{P}}_{zk}}{{\underline{D}}_{a}^{- 1} {\underline{P}}_{zk} {\underline{D}}_{b}^{- 1} {\underline{P}}_{yk}}$$

Assuming the underlying blood at each site is uniform, for example, this ratio may remove all weights of the site and the individual. One can see this explicitly if the underlying set of analytes are orthonormal. Under those conditions, the analyte inner product vector D may have just one non-zero element, and so the matrix math may collapse into a ratio of inner products:

$$\begin{matrix}{R}_{abyzkk} = \frac{( {c}_{y} {c}_{k} {\sum }_{i = 1}^{N} {c}_{i} {a}_{i}^{\to } \cdot {a}_{a}^{\to } ) ( {c}_{z} {c}_{k} {\sum }_{i = 1}^{N} {c}_{i} {a}_{i}^{\to } \cdot {a}_{b}^{\to } )}{( {c}_{z} {c}_{k} {\sum }_{i = 1}^{N} {c}_{i} {a}_{i}^{\to } \cdot {a}_{a}^{\to } ) ( {c}_{y} {c}_{k} {\sum }_{i = 1}^{N} {c}_{i} {a}_{i}^{\to } \cdot {a}_{b}^{\to } )} \\ = \frac{( {\sum }_{i = 1}^{N} {c}_{i} {a}_{i}^{\to } \cdot {a}_{a}^{\to } ) ( {\sum }_{i = 1}^{N} {c}_{i} {a}_{i}^{\to } \cdot {a}_{b}^{\to } )}{( {\sum }_{i = 1}^{N} {c}_{i} {a}_{i}^{\to } \cdot {a}_{a}^{\to } ) ( {\sum }_{i = 1}^{N} {c}_{i} {a}_{i}^{\to } \cdot {a}_{b}^{\to } )} \\ = 1\end{matrix}$$

The simplification shown here may note that, as long as the underlying blood is the same mixture of analytes, these ratios may all deliver the same values. Any differences may be due to systematic errors in the data or the data processing. Thus, when comparing measurements between many sites, one may have a quantitative score on how consistent the measurements will be. For N analytes, there may thus be N(N−1)/2 individual relationships like this which one may consider. Because there are so many relationships, one may write these relationships as a set of equations. And using that set of equations, one may minimize the systematic error created by applying the wrong calibration to the dataset. This may allow one to iteratively estimate the correct calibration and/or a minimum mean square methods. This may be particularly valuable for various embodiments that may make use of multiple sites.

Some embodiments provide for self-calibration on data between multiple individuals. For example, when one measures the same analyte at different sites between different hosts, one may examine a different relationship:

$${R}_{aayzkl} = \frac{{C}_{ayk} {C}_{azl}}{{C}_{azk} {C}_{ayl}} = \frac{{\underline{D}}_{a}^{- 1} {\underline{P}}_{yk} {\underline{D}}_{a}^{- 1} {\underline{P}}_{zl}}{{\underline{D}}_{a}^{- 1} {\underline{P}}_{zk} {\underline{D}}_{a}^{- 1} {\underline{P}}_{yl}}$$

This may be projected onto an orthonormal basis:

$$\begin{matrix}{R}_{aayzkl} = \frac{( {c}_{y} {c}_{k} {\sum }_{i = 1}^{N} {c}_{i} {a}_{i}^{\to } \cdot {a}_{a}^{\to } ) ( {c}_{z} {c}_{k} {\sum }_{i = 1}^{N} {c}_{i} {a}_{i}^{\to } \cdot {a}_{a}^{\to } )}{( {c}_{z} {c}_{k} {\sum }_{i = 1}^{N} {c}_{i} {a}_{i}^{\to } \cdot {a}_{a}^{\to } ) ( {c}_{y} {c}_{l} {\sum }_{i = 1}^{N} {c}_{i} {a}_{i}^{\to } \cdot {a}_{a}^{\to } )} \\ = 1\end{matrix}$$

This method may set up the same relationship between individuals as used above. If the bloodwork was identical between multiple sites, these relationships may hold. As it happens, some analytes may actually vary from one site to the next; the left arm vs the right arm may have shown glucose level differences of 8 mg/dL, for example. But for most substances, site differences are generally sufficiently small. Such ratios may allow one to write a subset of equations, and using these equations, one may minimize the systematic errors introduced by an improper calibration. Thus, one may reduce the impact of the amplitude variation between individuals due to improper calibration.

Some embodiments provide for image-based calibration. For example, one may directly measure the difference in scattering and reflectivity using the imaging system built into the targeting device. By imaging the subject at a common band (like 785 nm), one may measure the scattering and reflectivity of a target vein and of the skin surface, for example. Such measurements may occur at least once (during a patient's first use) and may be repeated for multi-patient installations. By comparing such a result to the reference dataset, one may adjust the calibration to match each patient and by incorporating the slope of the background scattering, one may have adjusted the models to fit the patient.

FIG. 8B shows an infrared image and graph 810 of a skin scene on the back of the hand, along with a slice through the image, in accordance with various embodiments as they related to the various methods disclosed, such as method 800. At 785 nm, for example, the light generally penetrates through the skin and is absorbed by the veins. Between the skin and the vein, it is generally scattered. When the image is calibrated, the trough of the darkest vein generally provides a measurement of the skin surface reflectivity. The bright skin between veins generally provides a measurement of the skin reflectivity plus the deep tissue scattering. Such an image may be formed on a targeting image sensor. The locations of veins beneath the skin may be used to control the first spatial light modulator in order to direct the illumination beam onto the vein. In these images, the position of the illumination beam may be used to control a second spatial light modulator and may enhance the light coupled into the entrance pupil of the spectrometer. When one measures the image at our targeted location, one generally gets a relative measurement of the reflectivity plus scattering which was presented to the Raman data. The skin reflectivity and scattering of the subject may then be compared to the model assumptions; this may allow one to adjust the signal level before processing the data. Doing so may correct for subject variations in methods generally discussed. Thus, the imaging hardware for targeting also may be used to estimate the skin reflectivity and the relative tissue scattering for a given targeted vein.

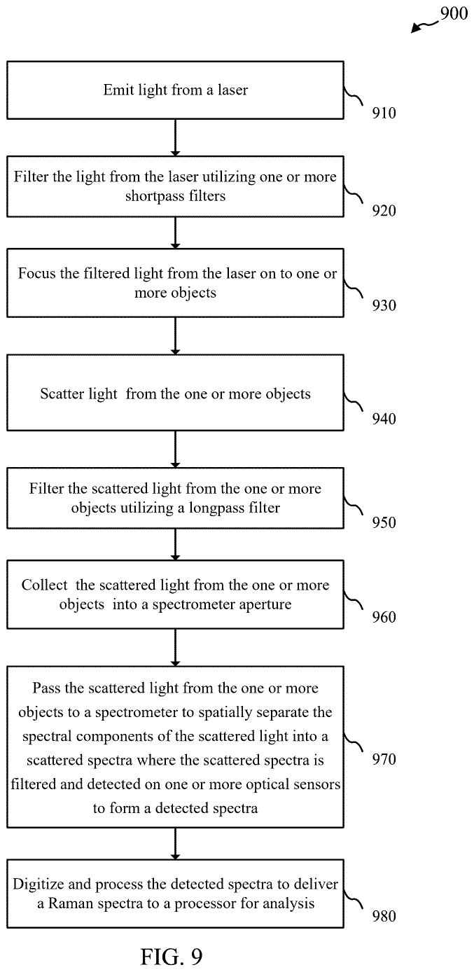

FIG. 9 provides a flow diagram of a method 900 in accordance with various embodiments. Method 900 may be implemented utilizing a variety of systems and/or devices shown and/or described with respect to FIG. 1, FIGS. 2, FIG. 3, FIGS. 4, FIG. 5, and/or FIG. 6. Method 900 may be an example of aspects of method 700 of FIG. 7 and/or method 800 of FIG. 8.

At block 910, light may be emitted from a laser. At block 920, the light from the laser may be filtered utilizing one or more short pass filters. At block 930, the filtered light from the laser may be focused on to one or more objects. At block 940, light may scatter from the one or more objects. At block 950, scattered light from the one or more objects may be filtered utilizing a long pass filter. At block 960, the scattered light from the one or more objects may be collected into a spectrometer aperture. At block 970, the scattered light from the one or more objects may pass through a spectrometer to spatially separate the spectral components of the scattered light into a scattered spectra; the scattered spectra may filtered and detected on one or more optical sensors to form a detected spectra. At block 980, the detected spectra may be digitized and processed to deliver a Raman spectra to a processor for analysis.

Some embodiments of the method 900 include beam steering the light from the laser utilizing one or more first spatial light modulators. Utilizing one or more first spatial light modulators may include utilizing one or more MEMs mirrors. Some embodiments further include beam shaping the scattered light from the one or more objects utilizing one or more second spatial light modulators.

Some embodiments of the method 900 include placing one or more body parts as the one or more objects and collecting the Raman spectra from the one or more body parts. Some embodiments include moving the one or more body parts in response to an indicator. The indicator may be proportional to one or more analyte measurements calculated from the Raman spectra. The one or more analyte measurements may include a hemoglobin measurement. Some embodiments include utilizing a targeting image sensor and an imaging system such that an image of the one or more body parts forms on the targeting image sensor and an image from the image sensor is communicated to the processor to control a MEMS mirror; the indicator may be a measured overlap of the illumination beam with a vein of the human hand as measured using the targeting image sensor. Some embodiments include measuring an analyte using the Raman spectra when one or more analyte measurements fall within a specific range.

Some embodiments of method 900 include processing the Raman spectra that may include: removing background scattered light from the Raman spectra to produce a bias-free spectra; calculating two or more coarse analyte measurements as an inner product of the bias-free spectra with two or more reference analyte spectra; and calculating two or more refined analyte measurements by compensating for multiple analyte inner products. Compensating for the multiple analyte inner products may include: loading the two or more coarse analyte measurements into a vector; and multiplying the vector by an inverse of a reference inner product matrix within the two or more reference analyte spectra.

Some embodiments of the method 900 include determining if one or more of the refined analyte measurements are sufficient. The one or more values of the one or more refined analyte measurements may be utilized to control a collection of the Raman spectra. Calculating the two or more coarse analyte measurements may include: modeling the Raman spectra as a weighted sum of a reference spectra; and estimating a plurality of weights that deliver a minimum mean square estimate of the bias-free spectra.

Some embodiments of the method include processing the Raman spectra that may include processing two or more digital spectra to produce two or more sets of refined analyte measurements. Some embodiments include: calculating one or more amplitude closure values from the two or more sets of refined analyte measurements, and calculating one or more errors in one or more amplitude closure values by multiplying the amplitude closure values by a pseudoinverse of an amplitude closure matrix. Some embodiments correct the two or more sets of refined analyte measurements by removing the errors. This result may be deemed one example of a self-calibrated analyte measurement.

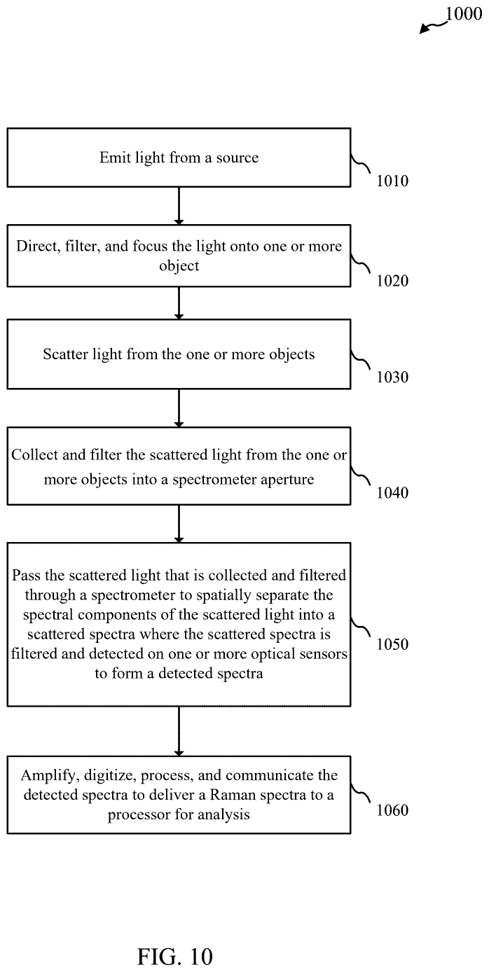

FIG. 10 provides a flow diagram of a method 1000 in accordance with various embodiments. Method 1000 may be implemented utilizing a variety of systems and/or devices shown and/or described with respect to FIG. 1, FIGS. 2, FIG. 3, FIGS. 4, FIG. 5, and/or FIG. 6. Method 1000 may be an example of aspects of method 700 of FIG. 7, method 800 of FIG. 8, and/or method 900 of FIG. 9.

At block 1010, light may be emitted from a source. At block 1020, the light may be directed, filtered, and focused onto one or more objects. At block 1030, light from the one or more objects may be scattered. At block 1040, the scattered light from the one or more objects may be collected and filtered into a spectrometer aperture. At block 1050, the scattered light that is collected and filtered may pass through a spectrometer to spatially separate the spectral components of the scattered light into a scattered spectra where the scattered spectra is filtered and detected on one or more optical sensors to form a detected spectra. At block 1060, the detected spectra may be amplified, digitized, processed, and communicated to deliver a Raman spectra to a processor for analysis.

In some embodiments of the method 1000, directing the light onto the one or more objects utilizes one or more spatial light modulators. Some embodiments include positioning the one or more spatial light modulators in a Fourier plane of the one or more objects with a Fourier optical system such that a beam angle produced by the one or more spatial light modulators shifts one or more positions at which one or more beams is focused on the one or more objects. In some embodiments, the source includes a multi-mode source and the one or more spatial light modulators are placed in a Fourier plane of the one or more objects within a Fourier optical system such that beam angle produced by each of the one or more spatial light modulators shifts one or more positions at which two or more beams are focused on the one or more objects. In some embodiments, the one or more spatial light modulators include one or more MEMs mirrors.

Some embodiments of the method 1000 include forming an image of the one or more objects on a targeting imaging sensor. Some embodiments include controlling the one or more spatial light modulators utilizing the image of the one or more objects on the targeting image sensor.

In some embodiments of the method 1000, the 4-F Fourier optical system includes a concave mirror that includes a conic curvature. In some embodiments, the one or more spatial light modulators and the targeting image sensor are mounted on a planar surface. In some embodiments, the one or more optical sensors and the diffractor of the spectrometer are co-planar and/or on another planar surface.

Some embodiments of the method 1000 include utilizing an analyte measurement method that processes the digitized spectra to deliver one or more analyte measurements. The one or more spatial light modulators may be controlled to maximize one or more analyte measurements.

Some embodiments of the method 1000 include beam shaping the scattered light utilizing a spatial light modulator. The spatial light modulator may be positioned in the Fourier plane of the one or more objects such that each spectral frequency of the spatial light modulator diffracts light to a resolvable position in a plane of the spectrometer aperture of the spectrometer. Some embodiments include utilizing an analyte measurement method that processes the digitized spectra to deliver one or more analyte measurements, wherein the spatial light modulator is controlled to maximize one or more analyte measurements. Some embodiments of the method include beam shaping the scattered light utilizing an array of two or more MEMS mirrors.

In some embodiments of the method 1000, a Fourier optical system is constructed utilizing a concave mirror that is hyperbolic. In some embodiments, three or more of the spatial light modulator, the two or more MEMS mirrors, and the targeting image sensor are co-planar. In some embodiments, the optical sensor and the diffractor of the spectrometer are co-planar with the source.

Some embodiments of the method 1000 include: placing a human hand as the one or more objects; and collecting the Raman spectra from the human hand. Some embodiments include moving the human hand in response to an indicator. The indicator may be proportional to the one or more analyte measurements. The one or more analyte measurements may include a hemoglobin measurement. Some embodiments include utilizing a targeting image sensor and an imaging system such that an image of the human hand forms on the targeting image sensor and an image from the image sensor is communicated to the processor to control a MEMS mirror; the indicator may be a measured overlap of the illumination beam with a vein of the human hand as measured using the targeting image sensor.

Some embodiments of the method 1000 include processing the Raman spectra that includes: removing background scattered light from the Raman spectra to produce a bias-free spectra; calculating two or more coarse analyte measurements as an inner product of the bias-free spectra with two or more reference analyte spectra; and calculating two or more refined analyte measurements by compensating for multiple analyte inner products. Compensating for the multiple analyte inner products may include: loading the two or more coarse analyte measurements into a vector; and multiplying the vector by an inverse of a reference inner product matrix within the two or more reference analyte spectra. Some embodiments include analyzing the Raman spectra for one or more analytes to determine if one or more values of the one or more analytes are sufficient. The one or more values of the one or more analytes may be utilized to control a collection of the Raman spectra. Calculating the two or more coarse analyte measurements may include: modeling the Raman spectra as a weighted sum of a reference spectra; and estimating multiple weights that deliver a minimum mean square estimate of the bias-free spectra.

In some embodiments of the method 1000, processing the Raman spectra includes processing two or more digital spectra to produce two or more sets of refined analyte measurements. Some embodiments include: calculating one or more amplitude closure values from the two or more sets of refined analyte measurements, and calculating one or more errors in one or more amplitude closure values by multiplying the amplitude closure values by a pseudoinverse of an amplitude closure matrix. Some embodiments include correcting the two or more sets of refined analyte measurements by removing the one or more errors.

A wide variety of terms are utilized throughout the present disclosure. The following additional descriptions regarding some of these terms and is provided solely for the purpose of facilitating understanding of the present disclosure. Unless expressly stated otherwise, the inclusion of a term and its description herein is not intended to act as a lexicographic definition that limits the meaning of that term, the scope of the claims, or equivalents thereof.

A Fourier optical system is used several times within the systems described in this invention. Whereas rays traverse all optical systems with a combination of ray height from the optical axis and angular deviation from the optical axis, in Fourier optical systems, there may be two or more related planes in the system where (i) the field height at a second plane corresponds to a mapping of the angular deviation of one or more rays in the first plane, (ii) the angular deviation in the second plane corresponds to a mapping of the field height in the first plane. When the mapping of the field height at the second plane corresponds sufficiently to the sine of the angular deviation of one or more rays in the first plane and the sine of the angular deviation in the second plane maps sufficiently close to the field height in the first plane, the transverse electromagnetic field in the second plane may be approximately a Fourier transform of the transverse electromagnetic plane in the first plane. Under this condition, one may refer to the second plane as the Fourier plane of the first plane. In some embodiments, the mapping of the field height at the second plane may correspond sufficiently to the tangent of the angular deviation of one or more rays in the first plane; in some embodiments, one may refer to the second plane as the Fourier plane of the first plane.

The relationship between a Fourier plane and a first plane may be created in several ways. In some embodiments, a lens is chosen with a focal length, a first plane is placed one focal length before said lens, and a Fourier plane is then produced one focal length behind said lens. In some embodiments, a concave mirror is chosen with a focal length, a first plane is placed one focal length before said lens, and a Fourier plane is then produced one focal length before said lens (and substantially co-located with the first plane). Both of the previous embodiments may be referred to as a 2-F Fourier optical system because they repeat the single focal length spacing two times. In some embodiments, one shortens this further and refer to these 2-F Fourier optical systems as 2-F systems.

When two 2-F Fourier optical systems are placed back-to-back, the result is generally referred to as a 4-F system where the output of the system may be an image of the input, and a Fourier plane may form at an intermediate position within the system. Such a system may be used as a relay optic. And, when the angle or the phase of the Fourier plane may be modified—as found in several embodiments within this specification—then the output position of the image may be modified.

The Fourier relationship may also be produced by, for example, placing two lenses with a focal length exactly one focal length apart; when the first plane is positioned in front of the first lens, then the Fourier plane results at the back. Such a system may be used to replace a 2-F Fourier optical system because it replicates the Fourier relationship. In this specification, we refer to said system as a 1-F system.

Diffractor is a term that may be used for any number of devices where a significant component of the output light is caused by diffraction of the input light, rather than refraction of the light or reflection of the light. Diffraction may occur when the output optical wavefront deviates from the input optical wavefront with sufficient spatial frequency that at least some of the light exhibits a resolvable deviation in the beam direction. Examples of diffractors include, but are not limited to, reflection gratings, transmission gratings, spatial light modulators, diffractive optical elements, or meta-optics. One might note that a mirror may produce diffraction at the edges or that a lens may produce diffraction at the edges, but beams interacting with the center of a mirror or lens may see insignificant diffraction. As a result, one does not generally refer to lenses or mirrors as diffractors.

Beam steering is generally a method by which one or more source beams may be moved with respect to one or more objects. The one or more beams may be collimated or the one or more beams may be focused. In some embodiments, beam steering is a change in the beam position. Other embodiments produce a change in the beam angle at the object or a change in both the beam angle and the beam position at the object. In some embodiments, beam steering is produced using a MEMS mirror placed in the Fourier plane of the object. In some embodiments, beam steering is performed by placing a spatial light modulator in the Fourier plane of the object.

Beam forming is generally a method by which the light from one or more objects is formed into one or more entrance apertures of the spectrometer. In some embodiments, beam forming is the modulation of the phase of the scattered light to control the properties of light passing through the entrance aperture of the spectrometer. In some embodiments, beam forming modulates the amplitude, modulates the polarization, modulates the phase along one polarization, and/or modulates a combination of amplitude, phase, and/or polarization. In some embodiments, beam forming is accomplished by a spatial light modulator placed in the Fourier plane of the object. In some embodiments, the spatial light modulator is placed elsewhere in the optical system.

Spectrums generally describe the wide array of systems and methods which separate two or more wavelengths of light to produce detectable amplitude and phase values at two or more spatial locations in a system. In one preferred embodiment, a spectrometer may be constructed by placing a grating in the Fourier plane of an entrance aperture, and by placing a detector array in the Fourier plane of the light diffracted from the spectrometer. In other embodiments, the spectrometer may be constructed of a refractive prism in place of the grating. In other embodiments, the entrance aperture may be one or more waveguides coupled to two or more micro-ring resonators feeding two or more detectors; in said embodiment, the two or more micro-ring resonators are tuned to two or more wavelengths such that the light at each detector is delivered at two or more wavelengths. In another embodiment, the entrance aperture may be one or more waveguides coupling to a holographic grating such that different wavelengths of light are diffracted at different angles; in said embodiment, placing a detector array in a Fourier plane of the grating may collect light from two or more wavelengths. In one embodiment, the light is generally spread across many detectors and involve substantial signal processing to deliver detectable amplitude or phase values at two or more wavelengths. In some embodiments, the Fourier plane of the entrance aperture may be produced by placing the entrance aperture one focal length from a concave mirror; the Fourier plane is one focal length from the mirror surface in the direction of the reflection. In some embodiments, the Fourier plane may be produced by replacing the concave mirror with a lens.

An entrance aperture of a spectrometer may be utilized to delineate the spectrometer from the steering and beamforming system. The structure, positioning, appearance, and utility of the aperture (which also may be referred to as a port) may vary depending on the spectrometer method.

As illumination light passes into the object, light is scattered from the object. This scattered spectra of this scattered light may include spectra of illumination light scattered with no spectral shift, the spectra of the Stokes shifted scattered light, and the spectra of anti-Stokes shifted light.

A detected spectra may generally refer to the filtered and spatially separated spectra which falls onto the detector array of the spectrometer. In some embodiments, the spectrometer has been calibrated such that the spectral value corresponding to each detector is known. In some embodiments, the detector array may be a two dimensional array where the resulting image may be post-processed using a method that may consolidate many detectors into one measurement at a single spectral value.

Raman spectra may generally involve being detected, digitized, processed, and stored within memory. A Raman spectra may be a collection of measured levels and the spectral value at which that measured values occur. In some embodiments, the Raman spectra has been calibrated and resampled such that the increment between each spectral value is uniform. In some embodiments, the spectral value is recorded as the wavenumbers relative to the calibrated wavelength, and said calibrated wavelength is generally equal to the illumination wavelength of the laser. In some embodiments, the Raman spectra is uncalibrated, and the calibration may be applied before proceeding with the following processing steps.

Bias free spectra may generally refer to the removal of the background from the Raman spectra to produce the bias-free spectra.

A spatial light modulator is referred to here as an SLM. This terminology is intended generally to describe a class of devices in which the field vector components at two or more positions may be multiplied by two or more distinct amplitudes, shifted by two or more distinct phases, or both. In a variation where two field vector components experience different modulation, the modulation may be considered polarization-dependent. The device may be reconfigured such that the modulation can change from one moment to the next. Examples of SLM may include, but are not limited to: a liquid crystal (LC) array, a liquid crystal on silicon (LCOS) array, an acousto-optic deflector (AOD), an array of electro-optic modulators (EOMs), a digital light processing (DLP) array, a tunable wedge, an array of tunable wedges, a tunable lens, an array of tunable lenses, a photorefractive crystal, an array of MEMS mirrors, a MEMS mirror, a movable photomask, a deformable mirror device (DMD) array, or a movable transparency.

A micro-electric mechanical system (MEMS) mirror may include one method by which a small mirror with tip, tilt, and/or piston capabilities can be added to a system. The small size of MEMS mirrors is generally recognized as an advantage in lower cost systems.

Targeting image sensor may include an image sensor that may be used to direct the beam steering. A detector array may include an image sensor placed at the spectrometer output.

In the field of optics, one class of systems is a Fourier optical system in which a lens may be placed one focal length from an object and the image may be placed one focal length from the lens. In such a system, optical beams from each position on the object may produce optical beams at a well-defined angle at the image plane. In such a system, optical beams from each angle may pass through a well-defined position at the object plane. In some embodiments, the position in the image plane is proportional to the sine of the angle at the object plane and the sine of the angle in the image plane is proportional to the position in the object plane. In other embodiments, the positions are proportional to the tangent of the angle. In yet other embodiments, the position is approximate and compensated by post-processing the result. In the embodiment where the angles are proportional to the sine of the angle, the field in the image plane is the spatial Fourier transform of the field in the object plane. In this condition, one may refer to the image plane as the Fourier plane of the object. In some embodiments, one may refer to the image plane the Fourier plane of the object even when the embodiment has a position related to the tangent of the angle and where there are substantial deviations from the sine. Under this terminology, an object placed in the object plane may produce a Fourier plane of the optical fields from the object in the image plane. Under these conditions, one may describe the image plane as the Fourier plane of that specific object. For example, when a source is placed in the object plane, one may call the image plane the Fourier plane of the source. When a MEMS mirror is placed in the object plane, one may call the image plane the Fourier plane of the MEMS mirror; and the Fourier plane of the MEMS mirror may contain the spatial Fourier transform of the light reflected from the MEMS mirror. When a spatial light modulator is placed in the object plane, one may call the image plane the Fourier plane of the spatial light modulator; and the Fourier plane of the spatial light modulator may contain the spatial Fourier transform of the light reflected from the spatial light modulator. When a grating is placed in the object plane, one may call the image plane the Fourier plane of the grating; and the Fourier plane of the grating may contain the spatial Fourier transform of the light reflected from the grating. In this case, the light reflected from the grating also may include light which is diffracted at an angle proportional to the arc-sine of the ratio of the wavelength to the grating period; in the Fourier plane, the position of the beams may be proportional to the sine of this angle so the position of the beams may be proportional to the ratio of the wavelength to the grating period. This may be the principle of one embodiment of the spectrometer.

In some embodiments, a first concave mirror is used for (i) collimating the laser beam and (ii) the Fourier transform optic in the spectrometer. In some embodiments, a second concave mirror is used as the Fourier transform optic in the beam steering and beam forming portions of the system. In some embodiments, a third concave mirror is used to direct beams from the beam steering section onto the object, and then to collect beams from the object and direct them toward the beam forming section of the system.

A cross-inner-product or cross-analyte inner product may refer to the inner product of one analyte spectra with a second analyte spectra. When a measured spectra contains the superposition of spectra from many analytes, the inner product of the measured spectra with one analyte spectra may contain several cross-inner-product terms.

A reference analyte spectra or spectrum may refer to the one spectra for each analyte within the analyte spectra library. An analyte spectra library may refer to a collection of one spectra for each analyte used in the processing. The spectra may be measured, modeled, or theorized.

Modeling methods may refer to modeling the spectra as a weighted sum of the reference spectra and then estimating the weights which deliver the minimum mean square estimate of the measured bias-free spectra.

Inner product analyte measurement may refer to the inner product of the bias-free spectrum with the reference analyte spectrum.

Analyte measurement algorithm may refer to a measurement that may compensate for the cross-inner-product terms; this may also be referred to as a multi-analyte measurement algorithm. Analyte measurement compensated for cross-inner-product terms may refer to a measurement that may solve for the individual contribution of each analyte to the inner product analyte measurement by removing the cross-inner-product. Self-calibrated measurements may refer to an analyte measurement that may compensate for amplitude closure.

Amplitude closure may include a principle showing that, when measurements are taken in the presence of two gain terms, one may cancel the contributions of gain. For example, for every grouping of four gain terms (A, B, C, D), amplitude closure may collect four measurements of four gain terms that are present (AB, CD, AC, BD) and may calculate an amplitude closure value as the ratio: (AB times CD divided by AC times BD). The amplitude closure value may equal one because the four gain terms should cancel. Any deviation from one may be caused by a ratio of the errors in each measurement, thus, we may set up a set of equations such that we can solve for the errors in each measurement. Note; for these equations to be a linear set of equations, one may take the logarithm of the amplitude closure value and solve for the log of the measurement errors. One may refer to the log of the measurement errors as the amplitude closure errors, and these values may be subtracted from the measurements to calculate the log of the self-calibrated measurements.

A steering substrate may refer to a substrate may include many copies of the steering opto-electronics. This may include, but is not limited to, the MEMS, the mirrors, the targeting sensor, the SLM, etc. Steering integrated optic may refer to the delivery of one first concave mirror to each grouping of MEMS mirrors, mirrors, openings, and SLMs.

A spectrometer substrate may refer to a substrate that may include many copies of the spectrometer opto-electronics. This may include, but is not limited to, the source, the short pass filter, the long pass filter, the gratings, and/or the detector array. Spectrometer integrated optic may refer to one second concave mirror and one object mirror for each grouping of sources, gratings, long pass filters, short pass filters, and/or detector arrays. Substrate aperture may refer to an opening in the substrate through which light passes from the steering system to the object and/or the source.

A reflector may refer to anything in which the light leaving the surface is in the generally opposite direction. Elements which reflect light may include, but are not limited to: mirrors, reflective coatings, surface transparent objects, reflective spatial light modulators, and/or MEMS mirrors.

A spectrometer may refer to a device that may measure the light at one or more wavelengths of one or more beams of light passing into its input aperture. In some embodiments, a spectrometer is constructed of an input aperture, a first lens, a transmissive grating, a second lens, and a detector array where each element is separated by a focal length from the previous element; in this system, each detector in the detector array may measure the light within a specific spectral band corresponding to the location and width of the detector. Some embodiments of a spectrometer may replace one or more of the lenses with mirrors. Some embodiments may use a reflective grating rather than a transmissive grating. Some embodiments may use a single detector, and the detector may be moved in position. Some embodiments may tilt the grating to shift the span of spectral bands which fall on the detector array. In some embodiments, an spectrometer may include an input aperture, a first convex reflector, a reflective grating, a second convex reflector, and a detector array where each element is separated by a focal length from the previous element and the second convex reflector is a region on the first convex reflector.

A beam steering subsystem may include a MEMS mirror (or a spatial light modulator in general), the concave mirror, a reflector, another concave mirror, a processor, and the substrate aperture. Some embodiments include a targeting image sensor and an illumination source. Some embodiments include a spectrometer. A beam forming subsystem may include a spatial light modulator, a concave mirror, a reflector, another concave mirror, a processor, and a substrate aperture. Some embodiments include a targeting image sensor and an illumination source. Some embodiments include a spectrometer.

In some embodiments, a laser source includes a semiconductor laser, lenses to shape a beam, and/or a short pass filter. Some embodiments include a spatial filter to clean up the beam.

It should be noted that the methods, systems, and devices discussed above are intended merely to be examples. It must be stressed that various embodiments may omit, substitute, or add various procedures or components as appropriate. For instance, it should be appreciated that, in alternative embodiments, the methods may be performed in an order different from that described, and that various stages may be added, omitted, or combined. Also, features described with respect to certain embodiments may be combined in various other embodiments. Different aspects and elements of the embodiments may be combined in a similar manner. Also, it should be emphasized that technology evolves and, thus, many of the elements are exemplary in nature and should not be interpreted to limit the scope of the embodiments.

Specific details are given in the description to provide a thorough understanding of the embodiments. However, it will be understood by one of ordinary skill in the art that the embodiments may be practiced without these specific details. For example, well-known circuits, processes, algorithms, structures, and techniques have been shown without unnecessary detail in order to avoid obscuring the embodiments.

Also, it is noted that the embodiments may be described as a process which may be depicted as a flow diagram or block diagram or as stages. Although each may describe the operations as a sequential process, many of the operations can be performed in parallel or concurrently. In addition, the order of the operations may be rearranged. A process may have additional stages not included in the figures.

Having described several embodiments, it will be recognized by those of skill in the art that various modifications, alternative constructions, and equivalents may be used without departing from the spirit of the different embodiments. For example, the above elements may merely be a component of a larger system, wherein other rules may take precedence over or otherwise modify the application of the different embodiments. Also, a number of stages may be undertaken before, during, or after the above elements are considered. Accordingly, the above description should not be taken as limiting the scope of the different embodiments.

1. A method comprising:
emitting light from a laser;
filtering the light from the laser utilizing one or more short pass filters;
focusing the filtered light from the laser on to one or more objects;
scattering light from the one or more objects;
filtering the scattered light from the one or more objects utilizing a long pass filter;
collecting the scattered light from the one or more objects into a spectrometer aperture;
passing the scattered light from the one or more objects through a spectrometer to spatially separate the spectral components of the scattered light into a scattered spectra, wherein the scattered spectra is filtered and detected on one or more optical sensors to form a detected spectra; and
digitizing and processing the detected spectra to deliver a Raman spectra to a processor for analysis.

2. The method of claim 1, further comprising beam steering the light from the laser utilizing one or more first spatial light modulators.

3. The method of claim 2, wherein utilizing one or more first spatial light modulators includes utilizing one or more MEMs mirrors.

4. The method of claim 2, further comprising beam shaping the scattered light from the one or more objects utilizing one or more second spatial light modulators.

5. The method of claim 1, further comprising:
placing one or more body parts as the one or more objects; and
collecting the Raman spectra from the one or more body parts.

6. The method of claim 5, further comprising moving the one or more body parts in response to an indicator.

7. The method of claim 6, wherein the indicator is proportional to one or more analyte measurements calculated from the Raman spectra.

8. The method of claim 7, wherein the one or more analyte measurements includes a hemoglobin measurement.

9. The method of claim 6, further comprising:
utilizing a targeting image sensor and an imaging system such that an image of the one or more body parts forms on the targeting image sensor and an image from the image sensor is communicated to the processor to control a MEMS mirror, wherein the indicator is a measured overlap of the illumination beam with a vein of the human hand as measured using the targeting image sensor.

10. The method of claim 5, further comprising measuring an analyte using the Raman spectra when one or more analyte measurements fall within a specific range.

11. The method of claim 1, further comprising processing the Raman spectra that includes:
removing background scattered light from the Raman spectra to produce a bias-free spectra;
calculating two or more coarse analyte measurements as an inner product of the bias-free spectra with two or more reference analyte spectra; and
calculating two or more refined analyte measurements by compensating for a plurality of analyte inner products.

12. The method of claim 11, wherein compensating for the plurality of analyte inner products includes:
loading the two or more coarse analyte measurements into a vector; and
multiplying the vector by an inverse of a reference inner product matrix within the two or more reference analyte spectra.

13. The method of claim 11, further comprising determining if one or more of the refined analyte measurements are sufficient.

14. The method of claim 13, wherein the one or more values of the one or more refined analyte measurements are utilized to control a collection of the Raman spectra.

15. The method of claim 11, wherein calculating the two or more coarse analyte measurements includes:
modeling the Raman spectra as a weighted sum of a reference spectra; and
estimating a plurality of weights that deliver a minimum mean square estimate of the bias-free spectra.

16. The method of claim 11, wherein processing the Raman spectra includes processing two or more digital spectra to produce two or more sets of refined analyte measurements.

17. The method of claim 16, further comprising:
calculating one or more amplitude closure values from the two or more sets of refined analyte measurements,
calculating one or more errors in one or more amplitude closure values by multiplying the amplitude closure values by a pseudoinverse of an amplitude closure matrix; and
correcting the two or more sets of refined analyte measurements by removing the one or more errors.

18. A system comprising:
a laser, wherein light from the laser is collimated at a first focus to form a collimated laser light;
a first concave mirror positioned one focal length from the first focus such that the collimated laser light is focused by the first concave mirror to a second focus as a first converging light beam;
a short pass filter positioned in at least the collimated laser light or the first converging light beam;
one or more substrates positioned one focal length from the first concave mirror, wherein the one or more substrates include one or more substrate apertures such that the first converging light beam passes through at least one of the one or more substrate apertures;
a second concave mirror positioned two focal lengths from the first concave mirror such that the second concave mirror collimates the first converging light beam to a third focus as a second collimated light beam;
a MEMs mirror positioned at the third focus, wherein the MEMs mirror reflects the second collimated light beam back to the second concave mirror such that the second concave mirror focuses the second collimated light beam as a second converging light beam;
a first reflector positioned one focal length from the second concave mirror such that the first reflector reflects the second converging light beam off of the second concave mirror to a fourth focus within at least one of the one or more substrate apertures to form a third collimated light beam;
a third concave mirror positioned one focal length from the fourth focus, wherein the third concave mirror focuses light from the third collimated light beam into a third converging beam and directs the third converging beam to an object plane where one or more positions of the object scatter light back to the third concave mirror or a fourth concave mirror as one or more diverging object beams such that the one or more first diverging object beams are collimated by the third concave mirror or the fourth concave mirror to form one or more first collimated object beams that pass through at least one of the one or more substrate apertures and reflect off the second concave mirror or a fifth concave mirror to a first object beam focus to form one or more first converging object beams;
a second reflector positioned one focal length from the second concave mirror or the fifth concave mirror, wherein at least a portion of the second reflector is positioned at the first object beam focus such that the second reflector reflects the one or more first converging object beams from the first object beam focus as one or more second diverging object beams to the second concave mirror or the fifth concave mirror and the second concave mirror or the fifth concave mirror collimates the one or more second diverging object beams to produce one or more second collimated object beams;
a third reflector positioned one focal length from the second concave mirror or the fifth concave mirror, wherein the third reflector reflects the one or more second collimated object beams back at the second concave mirror or the fifth concave mirror such that the one or more second collimated object beams are focused to a second object beam focus within at least one of the one or more substrate apertures to form one or more third diverging object beams that are directed toward the first concave mirror or a sixth concave mirror such that the first concave mirror or the sixth concave mirror collimates the one or more third diverging object beams to form one or more third collimated object beams;
a first long pass filter positioned within the one or more third collimated object beams;
a diffractor positioned one focal length from the first concave mirror or the sixth concave mirror and intersecting a portion of the one or more third collimated object beams such that the one or more third collimated object beams diffracts according to a plurality of wavelengths of the one or more third collimated object beams and the plurality of wavelengths of the one or more third collimated object beams are directed at the first concave mirror or the sixth concave mirror such that a diffracted beam at each wavelength from the plurality of wavelengths is focused to a different position of the first concave mirror or the sixth concave mirror to form a spectra of focused beams;
a second long pass filter positioned in the spectra of focused beams; and
a detector array positioned in the spectra of focused beams such that the spectra of focused beams are detected, sampled, and communicated to a processor as a detected spectra.

19. (canceled)

20. The system of claim 18, wherein the second reflector is a spatial light modulator.

21.-29. (canceled)

30. The system of claim 18, wherein manufacturing the system includes dicing of a multi-layered wafer comprising:
a spectrometer integrated optic layer;
a spectrometer substrate layer;
a steering substrate layer; and
a steering integrated optic layer.

31.-75. (canceled)
